(19) United States
(12) Patent Application Publication
(10) Pub. No.: US 2008/0161365 A1
(43) Pub. Date: Jul. 3, 2008

(54) Methylene Derivatives

(75) Inventors: Richard J. Booth, Ann Arbor, MI (US); Patrick Michael O'Brien, Stockbridge, MI (US)

Correspondence Address: PFIZER INC., PATENT DEPARTMENT, MS8260-1611, GROTON, CT, 06340, US

(73) Assignee: PFIZER INC., Ann Arbor, MI (US)

(21) Appl. No.: 11/718,297

(22) PCT Filed: Dec. 5, 2005
(86) PCT No.: PCT/IB05/03769

§ 371 Date: Apr. 30, 2007

(51) Int. Cl. A61K 31/44; C07D 213/81; A61P 19/02

(52) U.S. Cl. 514/354; 546/323

(57) ABSTRACT

This invention relates to a compound of formula (I) or a pharmaceutically acceptable salt thereof; a pharmaceutical composition; a method of treating a disease mediated by an MMP-13 enzyme in a mammal; and a therapeutic combination containing at least two pharmaceutically active components, wherein R¹, Q, W¹, W², W^{2a}, R^{3a}, R^{3b}, L¹, and L², the pharmaceutical composition, the method of treating, and the therapeutic combination are as defined in the specification.

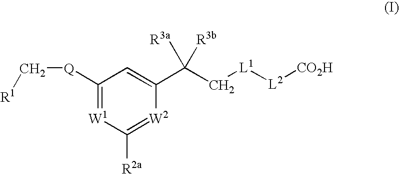

### FIELD OF THE INVENTION

The field of the present invention relates to compounds that are methylene derivatives; methods of using the compounds for treating diseases and disorders associated with MMP-13 mediated breakdown of extracellular matrix tissue; pharmaceutical compositions; and to combinations with other therapeutic agents.

### BACKGROUND OF THE INVENTION

Overactivity of matrix metalloproteinase-13 (“MMP-13”) in a mammal has been linked to the breakdown of collagen and bone of the joints in osteoarthritis and rheumatoid arthritis, breakdown of cartilage and bone in periodontitis, cardiac matrix tissue breakdown in heart failure, and tissue breakdown during invasive breast cancer tumor growth. Over expression of MMP-13 has been found in human squamous cell carcinomas of the larynx, head, and neck, human abdominal aortic aneurysm tissue, and atherosclerotic aorta tissue.

Potent and specific inhibitors of MMP-13 are desired for their ability to treat MMP-13 mediated diseases without stopping beneficial biological processes that are dependent upon the normal activity of one or more of the about 25 other MMP enzymes. This goal has become especially important in view of the musculoskeletal syndrome (“MSS”) side effect that has been observed during clinical trials with non-specific MMP inhibitors. PERIOSTAT® (doxycycline hyclate, Collagenex Pharmaceuticals, Inc., Newtown, Pa. 18940), an orally active compound that suppresses the production of MMP-8 and MMP13, was approved by the United States Food and Drug Administration in 2003 for treatment of gum disease.

U.S. Patent Application Publication Numbers 2002/0161000 A1, 2003/0144274 A1, 2003/0229103 A1, and 2004/0048863 A1 describe certain MMP-13 inhibitors.

There is still a need for MMP-13 inhibitors for treating osteoarthritis, rheumatoid arthritis, heart failure, breast cancer metastasis, and other diseases that are mediated, at least in part, by MMP-13 overactivity.

### BRIEF SUMMARY OF THE INVENTION

One aspect of the present invention is a compound of formula (I)

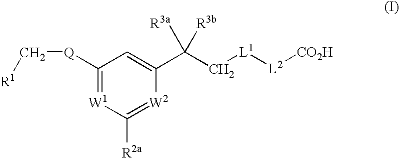

or a pharmaceutically acceptable salt thereof, wherein: R¹ is phenyl, or a 5- or 6-membered heteroatyl, wherein the phenyl, or 5- or 6-membered heteroaryl is unsubstituted or substituted on carbon atoms with from 1 to 3 substituent groups T¹; Q is —(H)N—C(═O)— or —C≡C—; W¹ and W² independently are N or C—R^{2b}; R^{2a} and each R^{2b} independently are H, F, C₁-C₃ alkyl, CF₃, —OH, —O—CH₃, —O—CF₃, —O—CH₂CH₃, or —NR^{2c}R^{2d}; or R^{2a} and R^{2b} are taken together to form a diradical —O—CH₂—O—; R^{2c} and R^{2d} independently are H, CH₃, or CH₂CH₃; R^{3a} is F and R^{3b} is H or F; or R^{3a} is OH and R^{3b} is H; or R^{3a} and R^{3b} are taken together with the carbon atom to which they are both attached to form C═N—OH; L¹ is CH₂, O, N(H), S, S(O), S(O)₂, CH₂CH₂, CH₂O, CH₂N(H), CH₂S, CH₂S(O), CH₂S(O)₂, OCH₂, N(H)CH₂, SCH₂, S(O)CH₂, or S(O)₂CH₂, wherein the nitrogen is optionally substituted with CH₃; L² is phenylene, 5- or 6-membered heteroarylene, C₅- or C₆-cycloalkylene, or a 5- or 6-membered heterocycloalkylene, wherein the phenylene or 5- or 6-membered heteroarylene is unsubstituted or substituted on carbon atoms with from 1 to 3 substituents independently selected from the group consisting of CH₃, —OH, —NH₂, F, and CF₃; wherein the C₅- or C₆-cycloalkylene or 5- or 6-membered heterocycloalkylene is unsubstituted or substituted on carbon atoms with from 1 to 3 substituents independently selected from the group consisting of CH₃, —OH, —NH₂, F, CF₃, and oxo; and wherein the 5-membered heteroarylene, 5- or 6-membered heterocycloalkylene is optionally substituted on a nitrogen atom with CH₃; and each T¹ independently is F, Cl, Br, I, —OH, —OCF₃, CF₃, —CN, —C₁-C₃ alkyl, —O—(C₁-C₃ alkyl), —C(O)—O—(C₁-C₃ alkyl), —C(O)—N(H)—(C₁-C₃ alkyl), or —S(O)₂—N(H)—(C₁-C₃ alkyl).

Another aspect of the present invention is a pharmaceutical composition, comprising the compound of formula (I), or a pharmaceutically acceptable salt thereof, and a pharmaceutic ally acceptable excipient.

Another aspect of the present invention is a method of treating osteoarthritis or rheumatoid arthritis in a mammal, the method comprising administering to a mammal in need thereof a therapeutically effective amount of the compound of formula (I), or a pharmaceutically acceptable salt thereof.

Another aspect of the present invention is the use of the compound of formula (I), or a pharmaceutically acceptable salt thereof, in the manufacture of a medicament for the treatment of osteoarthritis or rheumatoid arthritis in a mammal.

### DETAILED DESCRIPTION OF THE INVENTION

Terms used to describe a compound of formula (I), or a pharmaceutically acceptable salt thereof, are defined below.

The term “C₁-C₃ alkyl” means a straight or branched hydrocarbon chain having from 1 to 3 carbon atoms. Examples of C₁-C₃ alkyl groups include methyl, ethyl, 1-propyl, and 2-propyl.

“Phenylene” is 1,3- or 1,4-diradical of benzene.

A “5-membered heteroaryl” independently is a monocyclic, heteroaromatic ring radical that contains carbon atoms and from 1 to 4 heteroatoms independently selected from the group consisting of O, S, N, N(H), and N(CH₃). Examples of a 5-membered heteroaryl include thiophen-2-yl, furan-2-yl, pyrrol-3-yl, pyrazol-1-yl, imidazol-4-yl, isoxazol-3-yl, oxazol-2-yl, isothiazol-4-yl, thiazol-5-yl, [1,2,4]oxadiazol-3-yl, [1,3,4]thiadiazol-2-yl, [1,2,3]triazol-1-yl, [1,2,4]triazol-3-yl, tetrazol-1-yl, and the like. Preferred is isoxazolyl and oxazolyl.

A “6-membered heteroaryl” independently is a monocyclic, heteroaromatic ring radical that contains carbon atoms and 1 or 2 nitrogen atoms. Examples of a 6-membered heteroaryl include pyridin-4-yl, pyrimidin-2-yl, pyridazin-4-yl, pyrazin-2-yl, and the like. Preferred is pyridinyl.

A “5-membered heteroarylene” independently is a 5-membered monocyclic, heteroaromatic ring, 1,3-diradical containing carbon atoms and from 1 to 4 heteroatoms selected from the group consisting of O, S, N, N(H), and N(CH₃). Examples of a 5-membered heteroarylene include thiophen-2,5-diyl, furan-2,4-di-yl, pyrrol-1,3-di-yl, imidazol-1,4-diyl, tetrazol-2,5-diyl, [1,2,4]oxadiazol-3,5-diyl, [1,3,4]thiazol-2,5-diyl, and pyrazol-1,3-diyl.

A “6-membered heteroarylene” independently is a 6-membered monocyclic, heteroaromatic ring, 1,3- or 1,4-diradical containing carbon atoms and 1 or 2 N heteroatoms. Examples of a 6-membered heteroarylene include pyridin-2,5-diyl, pyrimidine-2,4-diyl, pyridazin-3,6-diyl, pyrazin-2,5-diyl, and pyrazin-2,6-diyl.

A “C₅- or C₆-cycloalkylene” independently is a monocyclic, hydrocarbon ring 1,3-diradical that contains 5 carbon atoms or a monocyclic 1,3- or 1,4-diradical ring that contains 6 carbon atoms, respectively, wherein the ring is saturated or optionally contains one carbon-carbon double bond. Examples of a C₅ or C₆ cycloalkylene include cyclopent-1,3-diyl, cyclopent-2-en-1,3-diyl, cyclohex-1,3-diyl, and cyclohex-1,4-diyl. Preferred is a cyclohex-1,4-diyl.

A “5- or 6-membered heterocycloalkylene” independently is a monocyclic ring 1,3-diradical that contains 5 ring atoms or a monocyclic ring 1,3- or 1,4-diradical that contains 6 ring atoms, respectively, wherein the ring atoms are carbon atoms and 1 or 2 heteroatoms independently selected from the group consisting of O, S, S(O), S(O)₂, N(H), and N(CH₃), and wherein when two heteroatoms are present, the two heteroatoms are not bonded to each other unless they comprise the group —S(O)₂—N(H)— or —S(O)₂—N(CH₃)—, and wherein the ring is saturated or optionally contains one carbon-carbon or carbon-nitrogen double bond. Examples of 5- or 6-membered heterocycloalkylene include tetrahydrofuran-2,4-diyl, morpholin-2,4-diyl, 2-thiacyclohex-1,4-diyl, 2-oxo-2-thiacyclohex-1,4-diyl, 2,2-dioxo-2-thiacyclohex-1,4-diyl, and piperazin-1,4-diyl.

Pharmaceutical compositions include homogeneous and heterogeneous mixtures of a compound of formula (I), or a pharmaceutically acceptable salt thereof, and a pharmaceutically acceptable excipient. Preferred is a substantially homogeneous mixture. Examples of pharmaceutically acceptable excipients include pharmaceutically acceptable diluents, carriers, and stabilizers. In addition to active ingredients and excipients, pharmaceutical compositions of the present invention may contain other components such as capsule shells, for example gelatin capsule shells.

The term “mammal” includes humans, companion animals such as cats and dogs, primates such as monkeys and chimpanzees, livestock animals such as horses, cows, pigs, and sheep, and laboratory animals such as rats, mice, guinea pigs, rabbits, hamsters, monkeys, dogs, cats, and transgenic mice. A preferred mammal is a human, dog, or cat. Most preferred is a human. The mammal may also be referred to herein as a “patient.”

A patient in need of treatment with a compound of the present invention is a patient at risk for getting a MMP-13 mediated disease or a patient having a MMP-13 mediated disease. A patient having a MMP-13 mediated disease may be identified in any of a number of ways such as by clinical diagnosis of the disease, by assaying blood or other fluid (e.g., joint synovial fluid or lymph) for levels of a biomarker evidencing the disease, by gross or histopathologic examination of a biopsied tissue of a suspected diseased organ, joint, or other body part, by imaging techniques such as nuclear magnetic resonance or x-ray imaging, or by assessing the patient for signs or symptoms of the disease, including, in some cases pain or inflammation or functional assessments such as joint movement or a cardiac stress test.

A patient at risk for a MMP-13 mediated disease may be characterized as having an elevated expression of MMP-13 in a tissue, a family history of the disease, a genetic marker for a predisposition to developing the disease, or a lifestyle that predisposes the patient to developing the disease.

For example, a patient at risk for osteoarthritis may be characterized as having an elevated expression of MMP-13 in a joint, a family history of osteoarthritis, a genetic marker for a predisposition to developing osteoarthritis, a person who is, was, or will be engaged in elite athletics or heavy labor such as foundry workers, or a person who is over 70 years of age. In the United States, a person over 70 years of age has nearly a 70% chance of showing radiographic evidence of osteoarthritis in at least one joint and virtually all persons over the age of 75 years have osteoarthritis in at least one joint.

A clinically measurable improvement of osteoarthritis includes radiographic evidence of a slowing or halting of joint space narrowing in a knee or hip joint, for example, with or without an improvement in a score from the WOMAC, Lequesne's functional index, PGIC, Likert or VAS diagnostic assessment. In vivo improvements in human and other mammalian patients include radiographic, biomarker, or histopathologic evidence of a slowing of disease progression compared to a control (joint or animal), halting of disease progression, and preventing the onset of disease progression in a patient at risk for osteoarthritis.

The phrase “treating”, which is related to the terms “treat” and “treated,” means successfully effecting an improvement of a disease according to a relevant method of the present invention. Such an improvement includes preventing, inhibiting, slowing, delaying onset, halting, or reversing the progression of the disease being treated and includes reducing the severity of a symptom such as pain and inhibiting extracellular matrix breakdown, and the like. Treating includes palliative and prophylactic effects.

Disease progression relates to disease pathology such as cartilage breakdown in osteoarthritis, extracellular matrix breakdown in a failing heart muscle, or extracellular matrix breakdown in blood vessel walls proximal to a primary tumor or distal from the primary tumor and proximal to a potential site of a secondary tumor. Disease progression also relates to symptoms such as joint pain, joint function, heart function, or tumor penetration.

The phrase “therapeutically effective amount” means an amount of a compound of the present invention, or a pharmaceutically acceptable salt thereof, that is sufficient to successfully treat a disease according to a relevant method of the present invention.

The term “drug(s)”, which is synonymous with the phrases “therapeutic agent,” therapeutically active component,” “active component(s)”, “active compound(s)”, and “active ingredient(s)”, includes, for example, a compound of formula (I), celecoxib, valdecoxib, parecoxib, NSAIDs, and the like, and pharmaceutically acceptable salts thereof.

The phrases “invention compound,” “compound of the present invention,” “compound of formula (I), or a pharmaceutically acceptable salt thereof” and the like mean a compound of formula (I), solvate, tautomer, isotope, geometric isomer, or stereoisomer thereof, or a pharmaceutically acceptable salt thereof, as defined herein.

While all of the compounds of the present invention are useful, certain invention compounds are particularly interesting and preferred. Individual preferred aspects of a compound of the present invention include a compound of formula (I), or a pharmaceutically acceptable salt thereof, having any one of the following limitations:
R¹ is a phenyl substituted with from 1 to 3 substituent groups T¹, wherein T¹ is as defined above for formula (I); R¹ is phenyl substituted by one F, CF₃, or OCH₃; R¹ is a 6-membered heteroaryl substituted on carbon atoms with from 1 to 3 substituent groups T¹, wherein T¹ is as defined above for formula (I); R¹ is a 6-membered heteroaryl that is pyridinyl or pyridinyl substituted on a carbon atom by OCH₃; Q is —(H)N—C(═O)—; Q is —C≡C—; W¹ is N and W² is C—R^{2b}; W¹ and W² are each C—R^{2b}; W¹ and W² are each N; R^{2a} and R^{2b} are each H; R^{2a} is CH₃; R^{2a} or R^{2b} is OCH₃; R^{2a} or R^{2b} is F; R^{2a} or R^{2b} is OH; R^{2a} or R^{2b} is N(CH₃)₂; R^{3a} is F and R^{3b} is H; R^{3a} and R^{3b} are each F; R^{3a} is OH and R^{3b} is H; L¹ is CH₂; L¹ is CH₂CH₂; L¹ is O; L¹ is N(H); L¹ is S(O)₂; T¹ is CF₃, OCH₃, or F; L² is 1,4-cyclohexylene; L² is 1,4-phenylene; or L² is 6-membered heteroarylene.

The functional groups of the below compounds of the Examples and the compounds named as preferred species below are preferred. Also preferred is a compound of formula (I), or a pharmaceutically acceptable salt thereof, wherein the definition of one of the nine groups R¹, Q, W¹, W², R^{2a}, R^{3a}, R^{3b}, L¹ and L² is selected from the group consisting of what is exemplified in the below compounds of the Examples and the compounds named as preferred species below, and the definitions of the remaining eight of the nine groups R¹, Q, W¹, W², R^{2a}, R^{3a}, R^{3b}, L¹, and L² are as defined above for formula (I).

More preferred is a compound of formula (I), or a pharmaceutically acceptable salt thereof, wherein R¹ is phenyl substituted with 1 or 2 substituents selected from the group consisting of F, —CF₃, —OCH₃, and CH₃ or a 6-membered heteroaryl that is pyridinyl substituted on a carbon atom with OCH₃, Q is —(H)N—C(═O)—, W¹ is N and W² is C—R^{2b}, R^{2a} is H or CH₃, and R^{2b} is H.

Also more preferred is a compound of formula (I), or a pharmaceutically acceptable salt thereof, wherein W¹ is N and W² is C—R^{2b}, R^{3a} is F and R^{3b} is H, and L¹ is CH₂, CH₂CH₂, or O.

Also more preferred is a compound of formula (I), or a pharmaceutically acceptable salt thereof, wherein W¹ is N and W² is C—R^{2b}, R^{3a} and R^{3b} are each F, and L¹ is CH₂, CH₂CH₂, or O.

Also more preferred is a compound of formula (I), or a pharmaceutically acceptable salt thereof, wherein W¹ is N and W² is C—R^{2b}, R^{3a} is F and R^{3b} is H, or R^{3a} and R^{3b} are each F, L¹ is CH₂ or O; and L² is 1,4-phenylene.

Also more preferred is a compound of formula (I), or a pharmaceutically acceptable salt thereof, wherein W¹ is N and W² is C—R^{2b}, R^{3a} is F and R^{3b} is H, or R^{3a} and R^{3b} are each F, L¹ is CH₂ or O; and L² is 1,4-cyclohexylene.

Also more preferred is a compound of formula (I), or a pharmaceutically acceptable salt thereof, wherein W¹ is N and W² is C—R^{2b}, R^{3a} is F and R³b is H, or R^{3a} and R^{3b} are each F, L¹ is CH₂ or O; and L² is 6-membered heteroarylene.

Still more preferred is a compound of the below Examples, or a pharmaceutically acceptable salt thereof.

Also still more preferred is a compound of formula (I) selected from the group consisting of:
4-{4,4-difluoro-4-[2-methyl-6-(3-trifluoromethyl-benzylcarbamoyl)-pyridin-4-yl]-butyl}-benzoic acid;
4-{3-fluoro-3-[2-(3-trifluoromethyl-benzylcarbamoyl)-pyridin-4-yl]-propyl}-benzoic acid;
4-{3,3-difluoro-3-[2-(3-trifluoromethyl-benzylcarbamoyl)-pyridin-4-yl]-propyl}-benzoic acid;
4-{3,3-difluoro-3-[2-methyl-6-(3-trifluoromethyl-benzylcarbamoyl)-pyridin-4-yl]-propyl}-benzoic acid;
4-{3-fluoro-3-[2-(3-fluoro-benzylcarbamoyl)-pyridin-4-yl]-propyl}-benzoic acid; and
4-{3,3-difluoro-3-[2-(3-fluoro-benzylcarbamoyl)-pyridin-4-yl]-propyl}-benzoic acid; or
a pharmaceutically acceptable salt thereof.

Also still more preferred is a compound of formula (I) selected from the group consisting of:
4-{2-fluoro-2-[2-(3-methoxy-benzylcarbamoyl)-pyridin-4-yl]-ethoxy}-benzoic acid; and
4-{2,2-difluoro-2-[2-methyl-6-(3-trifluoromethyl-benzylcarbamoyl)-pyridin-4-yl]-ethoxy}-benzoic acid; or
a pharmaceutically acceptable salt thereof.

Also still more preferred is a compound of formula (I) selected from the group consisting of:
5-{3-fluoro-3-[2-methyl-6-(3-trifluoromethyl-benzylcarbamoyl)-pyridin-4-yl]-propyl}-pyridine-2-carboxylic acid;
5-{3,3-difluoro-3-[2-methyl-6-(3-trifluoromethyl-benzylcarbamoyl)-pyridin-4-yl]-propyl}-pyridine-2-carboxylic acid;
5-{3-fluoro-3-[2-(3-fluoro-benzylcarbamoyl)-6-methyl-pyridin-4-yl]-propyl}-pyridine-2-carboxylic acid;
5-{3,3-difluoro-3-[2-(3-fluoro-benzylcarbamoyl)-6-methyl-pyridin-4-yl]-propyl}-pyridine-2-carboxylic acid;
5-{3-fluoro-3-[2-methyl-6-(3-trifluoromethyl-benzylcarbamoyl)-pyridin-4-yl]-propyl}-4-trifluoromethyl-pyridine-2-carboxylic acid;
5-{3-fluoro-3-[2-methyl-6-(3-fluoro-benzylcarbamoyl)-pyridin-4-yl]-propyl}-4-trifluoromethyl-pyridine-2-carboxylic acid;
5-{3,3-difluoro-3-[2-methyl-6-(3-trifluoromethyl-benzylcarbamoyl)-pyridin-4-yl]-propyl}-4-trifluoromethyl-pyridine-2-carboxylic acid; and
5-{3,3-difluoro-3-[2-methyl-6-(3-fluoro-benzylcarbamoyl)-pyridin-4-yl]-propyl}-4-trifluoromethyl-pyridine-2-carboxylic acid; or
a pharmaceutically acceptable salt thereof.

Also still more preferred is a compound of formula (I) selected from the group consisting of:
4-{3-fluoro-3-[2-(4-fluoro-benzylcarbamoyl)-6-methyl-pyridin-4-yl]-propyl}-cyclohexanecarboxylic acid;
4-{3,3-difluoro-3-[2-(4-fluoro-benzylcarbamoyl)-6-methyl-pyridin-4-yl]-propyl}-cyclohexanecarboxylic acid;
4-{3-fluoro-3-[2-methyl-6-(3-trifluoromethyl-benzylcarbamoyl)-pyridin-4-yl]-propyl}-cyclohexanecarboxylic acid; and
4-{3,3-difluoro-3-[2-methyl-6-(3-trifluoromethyl-benzylcabamoyl)-pyridin-4-yl]-propyl}-cyclohexanecarboxylic acid; or
a pharmaceutically acceptable salt thereof.

Also still more preferred is a compound of formula (I) selected from the group consisting of:
4-{3-fluoro-3-[2-(3-fluoro-benzylcarbamoyl)-6-methyl-pyridin-4-yl]-propyl}-3-trifluoromethyl-benzoic acid;
4-{3,3-difluoro-3-[2-(3-fluoro-benzylcarbamoyl)-6-methyl-pyridin-4-yl]-propyl}-3-trifluoromethyl-benzoic acid;
4-{3-fluoro-3-[2-methyl-6-(3-trifluoromethyl-benzylcarbamoyl)-pyridin-4-yl]-propyl}-3-trifluoromethyl-benzoic acid; and
4-{3,3-difluoro-3-[2-methyl-6-(3-trifluoromethyl-benzylcarbamoyl)-pyridin-4-yl]-propyl}-3-trifluoromethyl-benzoic acid; or
a pharmaceutically acceptable salt thereof.

Also still more preferred is a compound of formula (I) selected from the group consisting of:
4-{3-fluoro-3-[2-(3-methoxy-benzylcarbamoyl)-6-methyl-pyridin-4-yl]-propyl}-benzoic acid; and
4-{3,3-difluoro-3-[2-(3-methoxy-benzylcarbamoyl)-6-methyl-pyridin-4-yl]-propyl}-benzoic acid; or
a pharmaceutically acceptable salt thereof.

Also still more preferred is a compound of formula (I) selected from the group consisting of:
4-(3-fluoro-3-{2-methyl-6-[(pyridin-4-ylmethyl)-carbamoyl]-pyridin-4-yl}-propyl)-benzoic acid;
4-(3,3-difluoro-3-{2-methyl-6-[(pyridin-4-ylmethyl)-carbamoyl]-pyridin-4-yl}-propyl)-benzoic acid;
4-(3-fluoro-3-{2-[(2-methoxy-pyridin-4-ylmethyl)-carbamoyl]-6-methyl-pyridin-4-yl}-propyl)-benzoic acid; and
4-(3,3-difluoro-3-{2-[(2-methoxy-pyridin-4-ylmethyl)-carbamoyl]-6-methyl-pyridin-4-yl}-propyl)-benzoic acid; or
a pharmaceutically acceptable salt thereof.

Also still more preferred is a compound of formula (I) selected from the group consisting of:
4-{3-[2-(3,4-difluoro-benzylcarbamoyl)-6-methyl-pyridin-4-yl]-3-fluoro-propyl}-benzoic acid;
4-{3-[2-(3,4-difluoro-benzylcarbamoyl)-6-methyl-pyridin-4-yl]-3,3-difluoro-propyl}-benzoic acid; and
4-{3-[2-(3,4-difluoro-benzylcarbamoyl)-6-methyl-pyridin-4-yl]-3-fluoro-propyl}-cyclohexanecarboxylic acid; or
a pharmaceutically acceptable salt thereof.

Also still more preferred is a compound of formula (I) selected from the group consisting of:
4-(3-fluoro-3-{2-[3-(3-trifluoromethyl-phenyl)-prop-1-ynyl]-pyridin-4-yl}-propyl)-benzoic acid;
4-(3-fluoro-3-{2-[3-(4-fluoro-phenyl)-prop-1-ynyl]-6-methyl-pyridin-4-yl}-propyl)-benzoic acid;
4-(3,3-difluoro-3-{2-methyl-6-[3-(3-trifluoromethyl-phenyl)-prop-1-ynyl]-pyridin-4-yl}-propyl)-benzoic acid; and
4-(3,3-difluoro-3-{2-methyl-6-[3-(3-trifluoromethyl-phenyl)-prop-1-ynyl]-pyridin-4-yl}-propyl)-cyclohexanecarboxylic acid; or
a pharmaceutically acceptable salt thereof.

Also still more preferred is a compound of formula (I) selected from the group consisting of:
4-{2-[2-methyl-6-(3-trifluoromethyl-benzylcarbamoyl)-pyridin-4-yl]-2-hydroxy-ethoxy}-benzoic acid;
4-{3-[2-(3-methoxy-benzylcarbamoyl)-6-methyl-pyridin-4-yl]-3-hydroxy-propyl}-benzoic acid;
4-(3-{2-[3-(3-methoxy-phenyl)-prop-1-ynyl]-6-methyl-pyridin-4-yl}-3-hydroxy-propyl)-benzoic acid;
4-(3-{2-[3-(3-methoxy-phenyl)-prop-1-ynyl]-6-methyl-pyridin-4-yl}-3-hydroxy-propyl)-cyclohexanecarboxylic acid; and
4-{4-[2-methyl-6-(3-trifluoromethyl-benzylcarbamoyl)-pyridin-4-yl]-4-hydroxy-butyl}-benzoic acid; or
a pharmaceutically acceptable salt thereof.

Also more preferred is a compound of formula (I) selected from the group consisting of:
3-{3-[2,3-dimethyl-6-(3-trifluoromethyl-benzylcarbamoyl)-pyridin-4-yl]-3-hydroxyimino-propyl}-benzoic acid;
3-{2-hydroxyimino-2-[5-methoxy-2-(3-trifluoromethyl-benzylcarbamoyl)-pyridin-4-yl]-ethoxy}-cyclopentanecarboxylic acid;
5-(4-{6-[3-(3,4-difluoro-phenyl)-prop-1-ynyl]-3-methoxy-2-methyl-pyridin-4-yl}-4-hydroxyimino-butyl)-thiophene-2-carboxylic acid; and
3-(2-{5-[3-(4-fluoro-phenyl)-prop-1-ynyl]-[1,3]dioxolo[4,5-b]pyridin-7-yl}-2-hydroxyimino-ethanesulfonyl)-cyclohexanecarboxylic acid; or
a pharmaceutically acceptable salt thereof.

Also still more preferred is a compound of formula (I) selected from the group consisting of:
4-{3-fluoro-3-[4-methoxy-3-(3-trifluoromethyl-benzylcarbamoyl)-phenyl]-propyl}-benzoic acid;
3-(2,2-difluoro-2-{3-[3-(4-fluoro-phenyl)-prop-1-ynyl]-5-methyl-phenyl}-ethoxy)-cyclohexanecarboxylic acid; and
5-(3-hydroxy-3-{2,3,4-trifluoro-5-[(2-methoxy-pyridin-4-ylmethyl)-carbamoyl]-phenyl}-propyl)-isoxazole-3-carboxylic acid; or
a pharmaceutically acceptable salt thereof.

Also still more preferred is a compound of formula (I) selected from the group consisting of:
4-{3-fluoro-3-[6-(3-trifluoromethyl-benzylcarbamoyl)-pyrimidin-4-yl]-propyl}-benzoic acid;
4-{3-[2-dimethylamino-6-(3-trifluoromethyl-benzylcarbamoyl)-pyrimidin-4-yl]-3-fluoro-propyl}-benzoic acid;
4-(3,3-difluoro-3-{6-[3-(4-fluoro-phenyl)-prop-1-ynyl]-2-hydroxy-pyrimidin-4-yl}-propyl)-cyclohexanecarboxylic acid; and
1-(3-hydroxy-3-{6-[(2-methoxy-pyridin-4-ylmethyl)-carbamoyl]-2-methyl-pyrimidin-4-yl}-propyl)-pyrrolidine-3-carboxylic acid; or
a pharmaceutically acceptable salt thereof.

Some of the invention compounds are capable of forming pharmaceutically acceptable salts, including, but not limited to, acid addition and/or base salts. The acid addition salts are formed from basic invention compounds, whereas the base addition salts are formed from acidic invention compounds. All pharmaceutically acceptable salts are within the scope of the compounds useful in the invention.

Pharmaceutically acceptable acid addition salts of the basic invention compounds include salts derived from inorganic acids such as hydrochloric, nitric, phosphoric, sulfuric, hydrobromic, hydroiodic, hydrofluoric, phosphorous, and the like, as well as salts derived from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, malate, tartrate, methanesulfonate, and the like. Also contemplated are salts of amino acids such as arginate and the like and gluconate, galacturonate (see, for example, Berge S. M. et al., “Pharmaceutical Salts,” J. of Pharma. Sci., 1977;66:1).

Pharmaceutically acceptable base addition salts of the acidic invention compounds include salts derived from suitable metal cations such as sodium cation (Na⁺), potassium cation (K⁺), magnesium cation (Mg²⁺), calcium cation (Ca²⁺), and the like or suitable amines such as N,N′-dibenzylethylenediamine, chloroprocaine, choline, diethanolamine, dicyclohexylamine, ethylenediamine, N-methylglucamine, and procaine (see, for example, Berge, supra., 1977).

The free base forms of invention compounds differ from their respective acid addition salt forms somewhat in certain physical properties such as solubility, crystal structure, hygroscopicity, and the like, but the free base forms of the invention compounds and their respective acid addition salt forms are all useful for the purposes of the present invention.

The free acid forms of the invention compounds differ from their respective salt forms somewhat in certain physical properties such as solubility, crystal structure, hygroscopicity, and the like, but the salt forms and their respective free acid forms are all useful for purposes of the present invention.

Pharmaceutically acceptable base addition salts of invention compounds are preferred over pharmaceutically acceptable acid addition salts of basic invention compounds.

Invention compounds can exist in unsolvated forms as well as solvated forms, including hydrated forms and partially solvated forms (i.e., forms wherein the molar ratio of compound to solvent is not 1:1). The solvated forms and unsolvated forms are all encompassed within the scope of, and useful in, the present invention.

Certain of the invention compounds possess one or more chiral centers, and each chiral center may exist in the (R) or (S) configuration. An invention compound includes any stereoisomeric form of the compound, as well as mixtures thereof.

Additionally, certain invention compounds may exist as geometric isomers such as the entgegen (E) and zusammen (Z) isomers of alkenyl groups or cis and trans isomers of cycloalkyl groups. The invention includes any cis, trans, syn, anti, entgegen (E), or zusammen (Z) isomer of an invention compound, as well as any mixtures thereof.

Certain invention compounds can exist as two or more tautomeric forms. Tautomeric forms of the invention compounds may interchange, for example, via enolization/de-enolization, 1,2-hydride, 1,3-hydride, or 1,4-hydride shifts, and the like. The invention includes any tautomeric form of an invention compound, as well as any mixtures thereof.

The invention compounds also include isotopically-labelled compounds, which are identical to those recited above, but for the fact that one or more atoms are replaced by an atom having an atomic mass or mass number different from the atomic mass or mass number usually found in nature (i.e., different from the naturally abundant atomic mass or mass number). Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F and ³⁶Cl, respectively. Compounds of the present invention and pharmaceutically acceptable salts of the compounds which contain the aforementioned isotopes and/or other isotopes of other atoms are within the scope of this invention. Certain isotopically labelled compounds of the present invention, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of those described above in this invention can generally be prepared by carrying out the procedures incorporated by reference above or disclosed in the Schemes and/or in the Examples below, by substituting a readily available isotopically labelled reagent for a non-isotopically labelled reagent.

Compounds of formula (I), as defined above, may include certain compounds that contain an sp³-sp³ oxygen-oxygen, oxygen-nitrogen, oxygen-sulfur, nitrogen-nitrogen, nitrogen-sulfur, or sulfur-sulfur bond. Within these certain compounds is a first subset of compounds wherein the sp³-sp³ bond is to a nitrogen or sulfur atom and the nitrogen or sulfur atom is part of a functional group containing a carbon atom doubly bonded to the nitrogen atom (e.g., >C═N—O—) or an oxygen atom doubly bonded to the sulfur atom (e.g., —N(H)—S(═O)₂—, —O—S(═O)— or —O—S(═O)₂—). The certain compounds in this first subset are chemically and physically stable and are within the scope of the present invention. Also within these certain compounds, however, is a second subset of compounds, which are all the certain compounds that are not part of the first subset. The second subset of certain compounds may be chemically or physically unstable due to an art-recognized tendency of such bonds to break in the presence of, for example, oxygen or water, or upon heating or percussion, respectively. Accordingly, such certain compounds of formula (I) of the second subset of compounds are excluded from the present invention.

A compound of the present invention is particularly interesting and preferred if it has pharmacokinetic properties or aqueous solubility as described below, wherein the pharmacokinetics properties are as determined by Biological Example 4 and the aqueous solubility is as determined by Chemical Method 1. The pharmacokinetic properties include oral blood exposure as measured by area under the plasma drug concentration-time curve (“AUC”); an intravenous clearance rate of the compound from blood (“IV CL”); or half-life of the compound in blood (“T_{1/2}”).

Accordingly, another aspect of the present invention is a compound of formula (I), or a pharmaceutically acceptable salt thereof, that has an AUC, expressed in nanograms-hours per milliliter (“ng*hr/mL”) of >1000 ng*hr/ml after a single 5 mg/kg oral dose. Increasingly more preferred is an AUC after a single 5 mg/kg oral dose of >2000 ng*hr/mL, >5000 ng*hr/mL, and >10000 ng*hr/mL.

Another aspect of the present invention is a compound of formula (I), or a pharmaceutically acceptable salt thereof, that has an IV CL, expressed in milliliters per minute per kilogram of rat body weight (“mL/min/kg”), of <50, but >0.5, mL/min/kg after a single 5 mg/kg oral dose. Increasingly more preferred is an IV CL after a single 5 mg/kg oral dose of <40, but >0.5, mL/min/kg.

Another aspect of the present invention is a compound of formula (I), or a pharmaceutically acceptable salt thereof, that has a T_{1/2}, expressed in hours of >1, but <40 hours, after a single 5 mg/kg oral dose. Increasingly more preferred is a T_{1/2} after a single 5 mg/kg oral dose of >2, but <40, hours, >7, but <40, hours, >15, but <40, hours.

Another aspect of the present invention is a compound of formula (I), or a pharmaceutically acceptable salt thereof, that has an aqueous solubility, expressed in milligrams of compound per milliliter of solution (“mg/mL”), of >0.001 mg/mL. Increasingly more preferred is an aqueous solubility of >0.002 mg/mL, >0.01 mg/mL, or >0.02 mg/mL.

Another aspect of the present invention is a compound of formula (I), or a pharmaceutically acceptable salt thereof, that is a specific inhibitor of the enzyme MMP-13. A specific inhibitor of MMP-13, as used in the present invention, is a compound that is ≧5 times more potent in vitro versus MMP-13 than versus MMP-1, MMP-2, MMP-3, MMP-7, MMP-8, MMP-9, and MMP-14, wherein the potencies are determined according to the method of Biological Method 1. Increasingly preferred is a compound that is ≧10 times and ≧100 times more potent in vitro versus MMP-13 than versus MMP-1, MMP-2, MMP-3, 1MMP-7, MMP-8, MMP-9, and MMP-14.

Additional aspects of the present invention include individual methods for treating a disease, each individual method comprising administering to a mammal in need thereof a therapeutically effective amount of a compound of formula (I), or a pharmaceutically acceptable salt thereof wherein the disease is cartilage damage; heart failure; periodontitis; breast cancer; squamous cell carcinoma of the larynx; squamous cell carcinoma of the head; squamous cell carcinoma of the neck; abdominal aortic aneurysm; or atherosclerosis, respectively.

Another aspect of the present invention is a combination, comprising at least two therapeutically active components, which include a compound of formula (I), or a pharmaceutically acceptable salt thereof, together with at least one additional therapeutic agent. Preferred are combinations having only two therapeutically active components. Another aspect of the present invention is a pharmaceutical composition, comprising the invention combination, together with at least one pharmaceutically acceptable excipient. Another aspect of the present invention is a method of treating a MMP-13 mediated disease in a mammal, the method comprising administering to a mammal in need thereof an invention combination or an invention pharmaceutical composition.

A compound of the present invention may be combined with at least one of the various existing therapeutic agents that are known to treat the same diseases as those disclosed above. For the treatment of rheumatoid arthritis, a compound of the present invention may be preferably combined with at least one therapeutic biologic agent such as CP-870, etanercept, which is a tumor necrosis factor alpha (“TNF-alpha”) receptor immunoglobulin molecule, infliximab, which is an anti-TNF-alpha chimeric IgG 1K monoclonal antibody, or adalimumab, which is a human monoclonal anti-TNF-alpha antibody. Also preferably, a compound of the present invention may be combined with low dose methotrexate, lefunimide, hydroxychloroquine, d-penicillamine, auranofin or parenteral or oral gold to treat rheumatoid arthritis.

A compound of the present invention can also be used in combination with at least one of the existing therapeutic agents known to treat osteoarthritis or rheumatoid arthritis. Preferred existing therapeutic agents to be used in such combinations include non-steroidal anti-inflammatory agents (hereinafter NSAID's) such as piroxicam, diclofenac, propionic acids such as naproxen, flurbiprofen, fenoprofen, ketoprofen and ibuprofen, fenamates such as mefenamic acid, indomethacin, sulindac, apazone, pyrazolones such as phenylbutazone, salicylates such as aspirin, analgesics and intraarticular therapies such as corticosteroids, hyaluronic acids such as hyalgan and synvisc, and cyclooxygenase-2 (“COX-2”) inhibitors such as valdecoxib, celecoxib, parecoxib, etoricoxib, lumiracoxib, rofecoxib, tilacoxib, BMS-347070 (Chemical Abstracts Service Registry Number (“CAS Reg. No.”) [197438-48-5]), LAS-34475 (CAS Reg. No. [485397-26-0]), UR-8880 (CAS Reg. No. [265114-23-6]), ABT-963 (CAS Reg. No. [266320-83-6)), CS-502 [176429-82-6], (6aR,10aR)-3-(1,1-dimethylheptyl)-6a,7,10,10a-tetrahydro-1-hydroxy-6,6-dimethyl-6H-dibenzo[b,d]pyran-9-carboxylic acid (“CT-3”), CV-247 (CAS Reg. No. 1665026-43-7]), 2(5H)-furanone, 5,5-dimethyl-3-(1-methylethoxy)-4-[4-(methylsulfonyl)phenyl]- (“DFP”), GW-406381 (CAS Reg. No. [478702-57-7]), tiracoxib, meloxicam, nimesulide, 2-(acetyloxy)benzoic acid, 3-[(nitrooxy)methyl]phenyl ester (“NCX-4016”), P54 (CAS Reg. No. 130996-28-0), RevlMiD, 2,6-bis(1,1-dimethylethyl)-4-[(E)-(2-ethyl-1,1-dioxo-5-isothiazolidinylidene)methyl]phenol (“S-2474”), 5(R)-thio-6-sulfonamide-3(2H)-benzofuranone (“SVT-2016”), and N-[3-(formylamino)-4-oxo-6-phenoxy-4H-1-benzopyran-7-yl]-methanesulfonamide (“T-614”), Preferred COX-2 inhibitors include valdecoxib, celecoxib, and parecoxib.

Another aspect of the present invention is a combination, comprising a compound of formula (I), or a pharmaceutically acceptable salt thereof, and at least one therapeutically active agent in the below recited classes and under the following conditions:

A.) where a joint has become seriously inflamed as well as infected at the same time by bacteria, fungi, protozoa and/or virus, the invention compound is administered in combination with one or more antibiotic, antifungal, antiprotozoal and/or antiviral therapeutic agents;

B.) where a treatment of pain and inflammation is desired, the invention compound is administered in combination with inhibitors of mediators of inflammation, comprising one or more members independently selected from the group consisting essentially of:

(1) NSAIDs; (2) H₁-receptor antagonists; (3) kinin-B₁- and B₂-receptor antagonists; (4) prostaglandin inhibitors selected from the group consisting of PGD-, PGF-PGI₂- and PGE-receptor antagonists; (5) thromboxane A₂ (TXA₂-) inhibitors; (6) 5-, 12- and 15-lipoxygenase inhibitors, (7) leukotriene LTC₄-, LTD₄/TE₄- and LTB₄-inhibitors; (8) PAF-receptor antagonists; (9) gold in the form of an aurothio group together with one or more hydrophilic groups; (10) immunosuppressive agents selected from the group consisting of cyclosporine, azathioprine and methotrexate; (11) anti-inflammatory glucocorticoids; (12) penicillamine; (13) hydroxychloroquine; (14) anti-gout agents including colchicine; xanthine oxidase inhibitors including allopurinol; and uricosuric agents selected from probenecid, sulfinpyrazone and benzbromarone.

A compound of the present invention may be administered in combination with at least one therapeutic agent that is an inhibitor of one or more mediators of inflammation. The inhibitor is in a class of compounds selected from the group consisting essentially of matrix metalloproteinase inhibitors, aggrecanase inhibitors, TACE inhibitors, leucotriene receptor antagonists, IL-1 processing and release inhibitors, ILra, H₁-receptor antagonists; kinin-B₁- and B₂-receptor antagonists; prostaglandin inhibitors such as PGD-, PGF-PGI₂- and PGE-receptor antagonists; thromboxane A₂ (TXA2-) inhibitors, 5- and 12-lipoxygenase inhibitors; leukotriene LTC₄-, LTD₄/LTE₄- and LTB₄-inhibitors; PAF-receptor antagonists; gold in the form of an aurothio group together with various hydrophilic groups; immunosuppressive agents, e.g., cyclosporine, azathioprine and methotrexate; anti-inflammatory glucocorticoids; penicillamine; hydroxychloroquine; anti-gout agents, e.g., colchicine, xanthine oxidase inhibitors, e.g., allopurinol and uricosuric agents, e.g., probenecid, sulfinpyrazone and benzbromarone.

Where mammals are being treated for a cardiovascular disease which is heart failure, athrosclerotic aorta, or abdominal aortic aneurysm, the invention compound may be administered in combination with one or more members independently selected from the group consisting essentially of anti-hypertensives and other cardiovascular drugs intended to offset the consequences of atherosclerosis, hypertension, myocardial ischemia, angina, congestive heart failure and myocardial infarction, selected from the group consisting of: (1) a. diuretics; b. vasodilators; c. β-adrenergic receptor antagonists; d. angiotensin-II converting enzyme inhibitors (ACE-inhibitors), alone or optionally together with neutral endopeptidase inhibitors; e. angiotensin II receptor antagonists; f. renin inhibitors; g. calcium channel blockers; h. sympatholytic agents; i. α₂-adrenergic agonists; j. α-adrenergic receptor antagonists; and k. HMG-CoA-reductase inhibitors (anti-hypercholesterolemics); (2) antineoplastic agents selected from: a. antimitotic drugs selected from: i. vinca alkaloids selected from: [1] vinblastine and [2] vincristine; (3) growth hormone secretagogues; (4) strong analgesics; (5) local and systemic anesthetics; (6) H₂-receptor antagonists, proton pump inhibitors and other gastroprotective agents; (7) vasodilators such as hydralazine; (8) β-adrenergic receptor antagonists such as propranolol; (9) calcium channel blockers such as nifedipine; (10) α₂-adrenergic agonists such as clonidine; (11) α-adrenergic receptor antagonists such as prazosin; (12) HMG-CoA-reductase inhibitors (anti-hypercholesterolemics) such as lovastatin, simvastatin, rosuvastatin, or atorvastatin; (13) cholesterol ester transfer protein (“CETP”) inhibitors such as JTT-705 or CP-529,414; (14) Acyl coenzyme A: cholesterol acyl transferase (“ACAT”) inhibitors such as ezetimibe or avasimibe; or (15) microsomal triglyceride transfer protein (“MTTP”) such as implitapide.

Where a mammal is being treated for a cancer, a compound of the present invention may also be used in combination with at least one anticancer agent such as endostatin or angiostatin, or a cytotoxic drug such as adriamycin, daunomycin, cis-platin, etoposide, taxol, or taxotere, an alkaloid such as vincristine, or an antimetabolite such as methotrexate.

A compound of the present invention may also be used in combination with an anti-osteoporosis agent such as raloxifene, lasofoxifene, droloxifene or fosamax, or an immunosuppressant agent such as FK-506 or rapamycin, to treat bone degradation.

An invention compound, or a pharmaceutically acceptable salt thereof, or any therapeutic agent of an invention combination, may be formulated in dosage unit form with a pharmaceutically acceptable excipient. Some examples of dosage unit forms are tablets, capsules, pills, powders, aqueous and nonaqueous oral solutions and suspensions, and parenteral solutions packaged in containers containing either one or some larger number of dosage units and capable of being subdivided into individual doses.

Two or more active components of an invention combination may be formulated together in one capsule, tablet, intravenous solution, and the like or in independent separate formulations, or any combination thereof.

Formulations include controlled-release forms, which may contain a compound of the present invention alone or in a combination with another therapeutic agent as described herein. Controlled-release forms may be of particular usefulness when formulating an invention combination comprising a compound of the present invention and one or more other therapeutic agents which are to form an invention combination, wherein the compound and the therapeutic agents have varying half-lives. Controlled-release forms can be prepared that have different release times for the active ingredient(s), which achieves relatively uniform dosing. In the case of non-human patients, a medicated feed dosage form can be prepared in which active ingredients used in the combination are present together in admixture in a feed composition.

The percentage of a compound of the present invention in a pharmaceutical composition of the present invention can be varied within wide limits, but for practical purposes it is preferably at least 5% by weight in a solid composition and at least 2% by weight in a primary liquid composition. The most satisfactory compositions are those in which a much higher proportion of the active component(s) is present, for example, up to about 95% by weight. The tablets, powders, etc. of the invention composition typically contain from about 5% by weight to about 95% by weight of the total weight of the tablet, powder, etc., of the active component(s), preferably from about 5% to about 70% by weight.

Different routes of administration may require different dosages. For example, a useful intravenous (“IV”) dose is between 5 and 50 mg, and a useful oral dosage is between 20 and 800 mg, of a compound of formula (I), or a pharmaceutically acceptable salt thereof. The dosage is within the dosing range used in treatment of the diseases recited herein, or as would be routinely determined by a physician in accordance with the needs of the patient.

In therapeutic use as agents to treat the diseases listed herein, a compound of the present invention is administered to a patient at a dose that is effective for treating at least one symptom or pathology of the disease. The initial daily dosage of about 10 mg to about 2000 mg of the active component will typically be effective for an adult subject of normal weight. A daily dose range of about 10 mg to about 1000 mg of the active component is preferred and more preferred is from about 10 mg per day to about 500 mg per day.

A therapeutically effective amount of a compound of the present invention will generally be from about 0.02 mg/kg/dose to about 30 mg/kg/dose for an adult subject of normal weight, preferably from about 0.02 mg/kg/dose to about 15 mg/kg/dose.

In determining what constitutes a therapeutically effective amount, a number of factors will generally be considered by a physician or veterinarian in view of his or her experience. These factors include, for example, regulatory guidelines, including the Food and Drug Administration guidelines or guidelines from an equivalent agency, the results of published clinical studies, the particular mammal being treated, the patient's age, sex, weight and general health condition, as well as the type and extent of the disease being treated, and the use of other medications, if any, by the patient. Accordingly, the administered dose may fall within the ranges or concentrations recited herein, or may vary outside them, i.e., either below or above those ranges, depending, for example, upon the requirements of the individual patient, the severity of the condition being treated, and the particular therapeutic formulation being employed. Determination of a proper dose for a particular situation is routine and within the ordinary skill of the physician or veterinarian.

Generally, treatment may be initiated using smaller dosages of a therapeutic agent that are less than optimal for a particular patient. Thereafter, the dosage can be increased by small increments until an acceptable effect under the circumstance is reached. For convenience, the total daily dosage may be divided and administered in portions during the day, if desired.

Pharmaceutical compositions may be produced by formulating the compound of the present invention with a pharmaceutically acceptable excipient. Methods for preparing various pharmaceutical compositions with a certain amount of a therapeutic agent are known, or will be apparent and routinely determined in light of this disclosure, to those skilled in the art. For example, methods for preparing pharmaceutical compositions of the present invention may be adapted from those described in Remington's Pharmaceutical Sciences, Mack Publishing Company, Easton, Pa., 19^{th} edition (1995) and in the six-volume series, Handbook of Pharmaceutical Manufacturing Formulations, CRC Press, Boca Raton, Fla. (2004). When the formulations comprise the invention compound and a pharmaceutically acceptable excipient, they contain a therapeutically effective amount of the invention compound for the disease or disorder being treated.

Pharmaceutical compositions may or may not be in dosage unit form. Some examples of dosage unit forms are tablets, capsules, pills, powders, aqueous and nonaqueous oral solutions and suspensions, and parenteral solutions packaged in containers containing either one or some larger number of dosage units and capable of being subdivided into individual doses. Preferably such compositions are in unit dosage form.

In unit dosage form, a pharmaceutical preparation is subdivided into unit doses containing an appropriate quantity of the active component(s). The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Pharmaceutical compositions or “preparations” include the formulation of the active component(s) with encapsulating material such as a capsule shell, providing a capsule in which the active component(s), with or without other carriers, is surrounded by the capsule shell, which is thus in association with it.

Pharmaceutically acceptable excipients include sugars such as lactose and sucrose; starches such as corn starch and potato starch; cellulose derivatives such as sodium carboxymethyl cellulose, ethyl cellulose, methyl cellulose, and cellulose acetate phthalate; gelatin; talc; stearic acid; magnesium stearate; vegetable oils such as peanut oil, cottonseed oil, sesame oil, olive oil, corn oil, and oil of theobroma; propylene glycol, glycerin; sorbitol; polyethylene glycol; water; agar; alginic acid; isotonic saline, and phosphate buffer solutions; as well as other compatible substances normally used in pharmaceutical formulations.

The compositions to be employed in the present invention may also contain other components such as coloring agents, flavoring agents, and/or preservatives. These materials, if present, are usually used in relatively small amounts.

Suitable pharmaceutically acceptable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like.

The following formulation embodiment illustrates an invention pharmaceutical composition. The embodiment is representative only, and is not to be construed as limiting the invention in any respect.

FORMULATION EMBODIMENT 1
Tablet Formulation:

| Ingredient | Amount (mg) |
|---|---|
| An invention compound | 25 |
| Lactose | 50 |
| Cornstarch (for mix) | 10 |
| Cornstarch (paste) | 10 |
| Magnesium stearate (1%) | 5 |
| Total | 100 |

The invention compound, lactose, and cornstarch (for mix) are blended to uniformity. The cornstarch (for paste) is suspended in 200 mL of water and heated 5 with stirring to form a paste. The paste is used to granulate the mixed powders. The wet granules are passed thorough a No. 8 hand screen and dried at 80° C. The dry granules are lubricated with the 1% magnesium stearate and pressed into a tablet. Such tablets can be administered to a human from one to four times a day for treating a MMP-13 mediated disease.

A compound of the present invention can be prepared and administered according to a method of the present invention in a wide variety of oral and parenteral pharmaceutical dosage forms. Thus, the compound of the present invention can be administered by oral ingestion of a tablet, capsule, powder, solution and the like or by injection, that is, intravenously, intramuscularly, intracutaneously, subcutaneously, intraduodenally, or intraperitoneally. Also, the compound of the present invention can be administered by inhalation, for example, intranasally. Additionally, the compound of the present invention can be administered transdermally or by way of a suppository adapted for vaginal or rectal administration. It will be obvious to those skilled in the art that the following dosage forms may comprise as the active component a compound of the present invention.

Preferred routes of administration of a compound of the present invention are oral or by injection, such as injection to a joint. However, another route of administration may be preferred depending upon the particular condition being treated. Topical administration may be preferred for treating conditions localized to the skin. Topical administration by transdermal patch may also be preferred where, for example, it is desirable to effect sustained systemic dosing.

Solid form preparations are preferred. Solid form preparations include powders, tablets, pills, capsules, cachets, suppositories, and dispersible granules. A solid carrier can be one or more substances that may also act as diluents, flavoring agents, solubilizers, lubricants, suspending agents, binders, preservatives, tablet disintegrating agents, or an encapsulating material.

In powders, the carrier is a finely divided solid that is in a mixture with the finely divided active component. Powders suitable for intravenous administration or administration by injection may be lyophilized.

In tablets, the active component is mixed with the carrier having the necessary binding properties in suitable proportions and compacted in the shape and size desired.

Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogenous mixture is then poured into convenient sized molds, allowed to cool, and thereby to solidify.

Liquid form preparations include solutions, suspensions, and emulsions, for example, water or water propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizing, and thickening agents as desired.

Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations that are intended to be converted, shortly before use, to liquid form preparations for oral administration or injection. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

A preferred composition for human patients is a tablet or capsule form suitable for oral administration. Other preferred compositions for human patients provide delayed-, sustained- and/or controlled-release of an active ingredient. Such delayed-, sustained- and/or controlled-release compositions include all such dosage forms which produce ≧40% inhibition of disease sign or symptom or pathological result, and result in a plasma concentration of the active component of at least 2 fold higher than the active component's effective dose in 40% of patients (“ED₄₀”) for at least 8 hours; more preferably for at least 12 hours, more preferably still for at least 24 hours.

In veterinary use, a preferred composition for dogs comprises an ingestible liquid peroral dosage form selected from the group consisting of a solution, suspension, emulsion, inverse emulsion, elixir, extract, tincture and concentrate, optionally to be added to the food or drinking water of the dog being treated. Any of these liquid dosage forms, when formulated in accordance with methods well known in the art, can either be administered directly to the dog being treated, or may be added to the food or drinking water of the dog being treated. The concentrate liquid form, on the other hand, is formulated for dissolution in a given amount of water, from which solution an aliquot amount may be withdrawn for administration directly to the dog or addition to the dog's food or drinking water.

There is further provided in accordance with the present invention co-administration of a combination of drugs by the simultaneous or sequential administration of the drugs to be given in combination; including co-administration by means of different dosage forms and routes of administration; the use of combinations in accordance with different but regular and continuous dosing schedules whereby desired plasma levels of the drugs involved are maintained in the patient being treated, even though the individual drugs making up the combination are not being administered to the patient simultaneously.

A invention compound of formula (I), or a pharmaceutically acceptable salt thereof, and intermediates in the synthesis thereof may be prepared by one of ordinary skill in the art using routine synthetic chemistry methodology.

Syntheses of some invention compounds may utilize starting materials, intermediates, or reaction products that contain more than one reactive functional group. During chemical reactions, a reactive functional group may be protected from unwanted side reactions by a protecting group that renders the reactive functional group substantially inert to the reaction conditions employed. A protecting group is selectively introduced onto a starting material prior to carrying out the reaction step for which a protecting group is needed. Once the protecting group is no longer needed, the protecting group can be removed. It is well within the ordinary skill in the art to introduce protecting groups during a synthesis of a compound of formula (I), or a pharmaceutically acceptable salt thereof, and then later remove them. Procedures for introducing and removing protecting groups are known and referenced such as, for example, in Protective Groups in Organic Synthesis, 3^{rd} ed., Greene T. W. and Wuts P. G., Wiley-Interscience, New York, 1999, which is incorporated herein by reference.

Thus, for example, protecting groups such as the following may be utilized to protect amino, hydroxyl, and other groups: carboxylic acyl groups such as, for example, formyl, acetyl, and trifluoroacetyl; alkoxycarbonyl groups such as, for example, ethoxycarbonyl, tert-butoxycarbonyl (BOC), β,β,β-trichloroethoxycarbonyl (TCEC), and β-iodoethoxycarbonyl; aralkyloxycarbonyl groups such as, for example, benzyloxycarbonyl (CBZ), para-methoxybenzyloxycarbonyl, and 9-fluorenylmethyloxycarbonyl (FMOC); trialkylsilyl groups such as, for example, trimethylsilyl (TMS) and tert-butyldimethylsilyl (TBDMS); and other groups such as, for example, triphenylmethyl (trityl), tetrahydropyranyl, vinyloxycarbonyl, ortho-nitrophenylsulfenyl, diphenylphosphinyl, para-toluenesulfonyl (Ts), mesyl, trifluoromethanesulfonyl, and benzyl. Examples of procedures for removal of protecting groups include hydrogenolysis of CBZ groups using, for example, hydrogen gas at 50 psi in the presence of a hydrogenation catalyst such as 10% palladium on carbon, acidolysis of BOC groups using, for example, hydrogen chloride in dichloromethane, trifluoroacetic acid (TFA) in dichloromethane, and the like, reaction of silyl groups with fluoride ions, and reductive cleavage of TCEC groups with zinc metal.

In an illustrative method, compounds of formula (I) may be routinely prepared according to the synthetic routes outlined below in Schemes 1 to 3. In Scheme 1, commercially available 2-chlorosionicotinonitrile (Chemical Abstracts Registry number (“CAS Reg. No.”) [033252-30-1]) or 4-cyanopyridine (CAS Reg. No. [000100-48-1]) may be used to prepare a halo-substituted pyridinecarbonitrile such as (1). The halo-substituted pyridinecarbonitrile (1) is condensed with methyl magnesium bromide to give the corresponding methyl ketone (2). The methyl ketone (2) is condensed with carbon monoxide in the presence of a palladium catalyst and triethylamine to give an ethyl ester (3), which is condensed with a benzaldehyde (4) to give an enone (5). The enone (5) is reduced with hydrogen gas over palladium on barium sulfate to a mixture of a propanone (6) and propanol (7), which are separated by conventional means such as liquid chromatography, and the two intermediates are used separately in the next step as described for Scheme 2.

In Scheme 2, the propanone (6) or the propanol (7) is fluorinated with diethylaminosulfur trifluoride to give a difluoro-propane (8) or a monofluoro-propane (9), respectively. The difluoro-propane (8) or a monofluoro-propane (9) is then condensed with R¹CH₂NH₂ (10) in the presence of trimethylaluminum to give the corresponding amide-tertiary butyl ester (11) or (12), respectively. The amide-tertiary butyl ester (11) or (12) is deprotected with trifluoroacetic acid to give the carboxylic acid (13) or (14), respectively, which is a compound of formula (I).

In Scheme 3, the ethyl ester (3) is chlorinated with N-chlorosuccinimide (“NCS”) or brominated with N-bromosuccinimide (“NBS”) to give the alpha-chloro- or alpha-bromo-ketone (3a), respectively, which is condensed with a nucleophile (4a), wherein Y is O, S, or N(PG), wherein PG is an amine protecting group such as benzyl, to give ketone (5a). The ketone (5a) is reduced with a hydride reducing agent such as sodium borohydride to give alcohol (7a). Ketone (5a) or alcohol (7a) are fluorinated, condensed with R¹CH₂NH₂, and deprotected (including deprotection of PG) in a manner analogous to that described in Scheme 2 for ketone (6) and alcohol (7), respectively, to give a compound of formula (I).

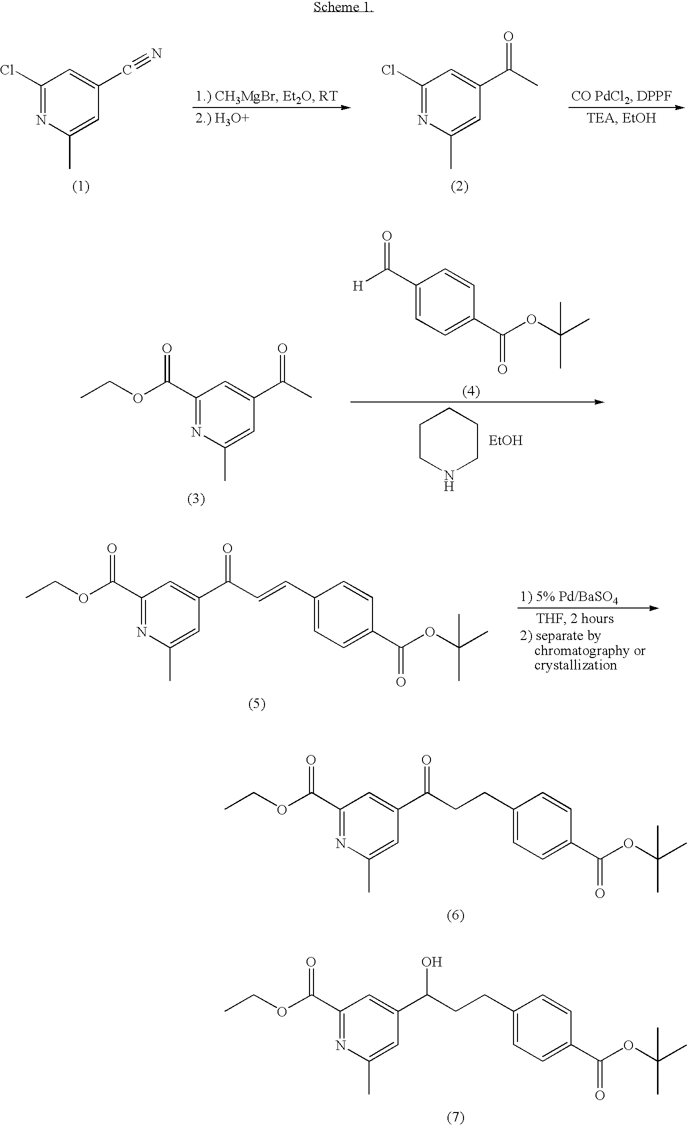

wherein Et₂O is diethyl ether, RT means room temperature, DPPF means 1,1′-bis(diphenylphosphino)ferrocene, TEA means triethylamine, EtOH means ethanol, THF means tetrahydrofuran.

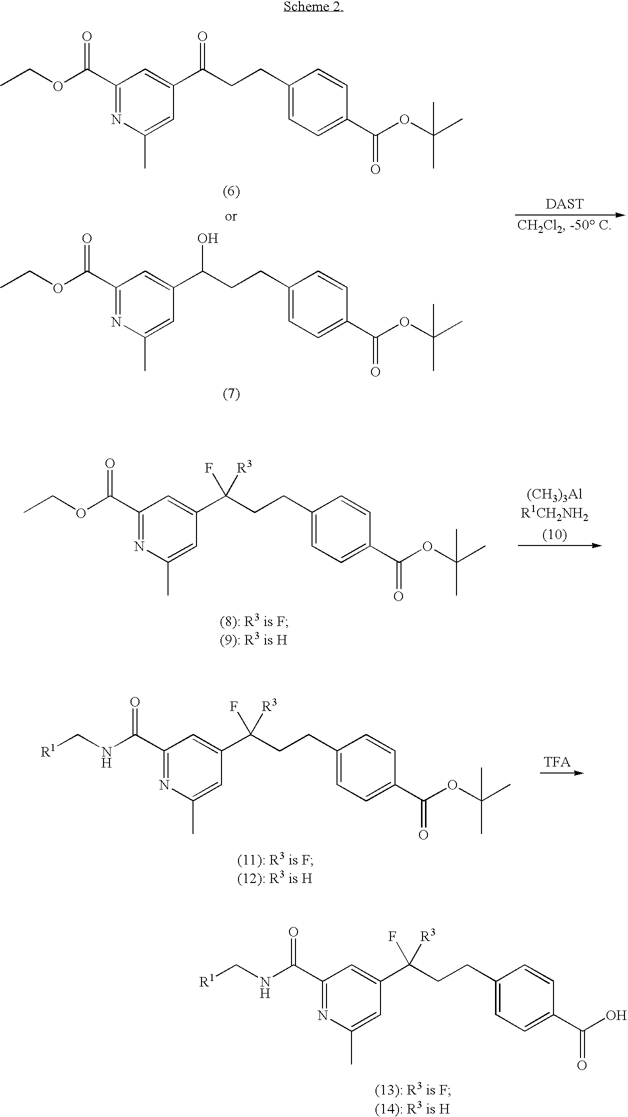

wherein DAST means diethylaminosulfur trifluoride, TFA means trifluoroacetic acid, and R¹ is as defined above for formula (I).

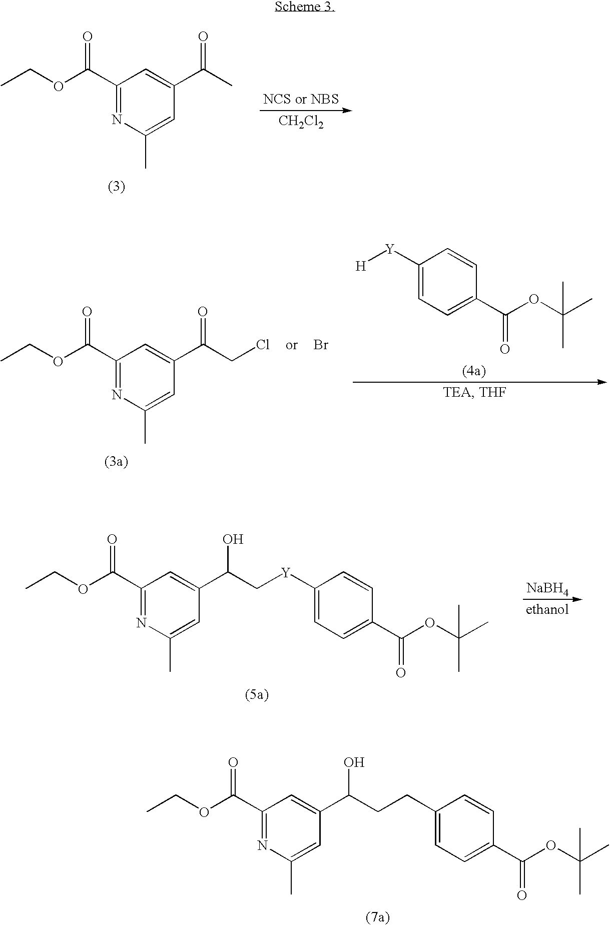

Alternatively a compound of formula (I) wherein Q is —C≡C— may be prepared by reacting 2-bromo-6-methyl-4-pyridine-carbonitrile, for example, with methyl magnesium bromide, in a manner analogous to the first reaction of Scheme 1, to give 4-acetyl-2-bromo-6-methylpyridine. The bromo of the 4-acetyl-2-bromo-6-methylpyridine can then be converted to a boronic acid (e.g., —B(OH)₂) derivative, and the boronic acid derivative reacted with an alkyne of formula R¹CH₂C≡CH under palladium-catalyzed coupling conditions such as “Suzuki” coupling conditions to give a compound of formula (D)

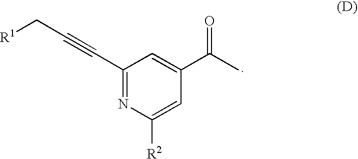

Alternatively, the 4-acetyl-2-bromo-6-methylpyridine can be directly coupled with a compound of formula R¹CH₂C≡CH under “Heck” arylation conditions using bis-[tris(ortho-methylphenyl)phosphine]palladium(II) chloride. The compound of formula (D) can be coupled with a compound of formula (4) in Scheme 1 to give an alkyne analog of the compound of formula (5) in Scheme 1 (i.e., the analog of the compound of formula (5) wherein the Q is —N(H)C(═O)— is replaced by Q is —C≡C—). Compounds of formula (I) wherein Q is —C≡C— can be prepared from the alkyne analog of the compound of formula (5) in a manner analogous to that illustrated in the remainder of Scheme 1 and in Schemes 2 or 3.

Alternatively, a compound of formula (I) wherein L¹ is CH₂O, CH₂N(H), CH₂S, CH₂S(O), or CH₂S(O)₂ may be prepared by reacting a compound of formula (E)

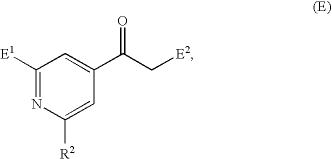

wherein E¹ is Br or CH₃CH₂OC(═O)— and E² is H, with N-chlorosuccinimide or N-bromosuccinimide to give a compound of formula (E) wherein E¹ is as defined above and E² is Cl or Br, respectively, which can in turn be reacted with a compound of formula (F) K¹—CH₂-L²-CO₂t-butyl (F), wherein K¹ is HO—, H₂N—, or HS—, to give a compound of formula (E) wherein E¹ is as defined above and E² is —O—CH₂-L²-CO₂t-butyl, —N(H)—CH₂-L²-CO₂t-butyl, or —S—CH₂-L²-CO₂t-butyl, respectively. The compound of formula (E) wherein E¹ is as defined above and E² is —S—CH₂-L²-CO₂t-butyl, can be treated with one or two mole equivalents of a mild oxidizing agent such as meta-chloroperbenzoic acid to give a compound of formula (E) wherein E¹ is as defined above and E² is —S(O)—CH₂-L²-CO₂t-butyl or —S(O)₂—CH₂-L²-CO₂t-butyl, respectively. Compounds of formula (E) wherein E¹ is as defined above and E² is —O—CH₂-L²-CO₂t-butyl, —N(H)—CH₂-L²-CO₂t-butyl, —S—CH₂-L²-CO₂t-butyl, —S(O)—CH₂-L²-CO₂t-butyl, or —S(O)₂—CH₂-L²-CO₂t-butyl, can be converted to a compound of formula (I) as illustrated in Schemes 1 and 2 or 3.

Alternatively compounds of formula (I) wherein L¹ is OCH₂, N(H)CH₂, SCH₂, S(O)CH₂, or S(O)₂CH₂ may be prepared by reacting an enolate of a compound of formula (E′)

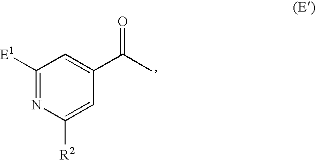

wherein E¹ is Br or CH₃CH₂OC(═O)— with a compound of formula (F′): BrCH₂—K¹-L²-CO₂t-butyl (F′), wherein K¹ is O, N(H), or S. The resulting intermediate can be treated with m-CPBA to give intermediates wherein K¹ is S(O) or S(O)₂. These intermediates can then be converted to a compound of formula (I) according to a procedure analogous to that illustrated in Schemes 1 and 2 or 3.

Alternatively, to prepare compounds of formula (I) wherein R^{3a} and R^{3b} are taken together with the carbon atom to which they are both bonded to form a C═N—OH, the corresponding ketone, such as compound (6) in Scheme 1 or compound (5a) in Scheme 3, are allowed to react with H₂N—OH.HCl in the presence of a non-nucleophilic base such as triethylamine or sodium hydride in a solvent such as toluene, tetrahydrofuran, or methanol, preferably in the presence of a dehydrating apparatus such as a Dean-Stark trap (toluene) or 3 Å molecular sieves. The resulting cis- and trans-oximes may be separated by chromatography or fractional crystallization.

One of ordinary skill in the art would know how to routinely prepare other compounds of formula (I), such as, for example, wherein W¹ and W² are each N or W¹ and W² are each C—R, by readily adapting the methods illustrated in Schemes 1 to 3 or described above. In the methods of Schemes 1 to 3 and related methods, solvents such as THF, dichloromethane, DMF, dioxane, diethyl ether, acetonitrile, ethanol, methanol, ethyl acetate, toluene, and the like are preferred unless otherwise indicated. Unless otherwise noted, preferred are reaction temperatures within a range of from about −80° C. to about 150° C., more preferably between −80° C. to about 100° C., and atmospheres over the reactions that are nitrogen or helium. The skilled artisan would routinely determine which solvents, reaction temperatures, and the like to use for a particular reaction. The solvents, reaction temperatures, atmospheres, and the like are not critical as long as some desired product is made therewith.

An acid addition salt of a basic invention compound may be prepared by contacting the free base form of the compound with a sufficient amount of a desired acid to produce a salt in a conventional manner. The free base form of the compound may be regenerated by contacting the acid addition salt with a base, and isolating the free base form of the compound in a conventional manner.

A pharmaceutically acceptable base addition salt of an acidic invention compound may be prepared by contacting the free acid form of the compound with a metal cation such as an alkali or alkaline earth metal cation, or an amine, especially an organic amine to produce the salt in a conventional manner. The free acid form of the compound may be regenerated by contacting the salt form with an acid, and isolating the free acid of the compound in a conventional manner.

Syntheses of certain compounds of formula (I) are described below in Examples 1 to 3.

### EXAMPLE 1

### Preparation of 4-{3-fluoro-3-[2-methyl-6-(3-trifluoromethyl-benzylcarbamoyl)-pyridin-4-yl]-propyl}-benzoic acid

### Step (1) Synthesis of 4-acetyl-6-methyl-pyridine-2-carboxylic acid ethyl ester

To a 300 mL high pressure reaction vessel were added 9.36 g (55.2 mmol, prepared as illustrated in Scheme 1) of 4-acetyl-2-chloro-6-methylpyridine, 0.451 g (0.552 mmol) of palladium (II) 1,1′-bis(diphenylphosphino)ferrocene (“Pd-DPPF”), 9.2 mL of triethylamine (66.2 mmol, 1.2 mole equivalents), and 140 mL of ethanol. The reaction vessel was sealed, placed in a stand, purged, and pressurized to 500 psi with carbon monoxide gas. Stirring was started and the reaction mixture was heated to 100° C. and stirred for a total of 14 hours. The reaction mixture was then cooled, the resulting solids were filtered off, and the filtercake was dissolved in methylene chloride. The insoluble solids (mostly triethylamine hydrochloride) were filtered off, and the filtrate was rotary evaporated over silica gel to give the silica gel with product adsorbed thereon. The product was purified in two equal lots on BIOTAGE® (Biotage, Inc., 1500 Avon Street Ext. Charlottesville Va., 22902) Flash 65i (350 g, silica gel) cartridges using a 30 minute gradient of from 50% to 75% (volume/volume, “v/v”) ethyl acetate in hexane. Product fractions from both columns were pooled and rotary evaporated, The resultant pale orange solid was dried under house high vacuum at 40° C. over about 3 days to give 10.5 g (92% yield) of 4-acetyl-6-methyl-pyridine-2-carboxylic acid ethyl ester, ¹H NMR (400 MHz, deuterated dimethylsulfoxide (“DMSO-D6”)) δ ppm 1.35 (t, J=7.20 Hz, 3H) 2.51 (s, 3H) 2.65 (s Hz, 3H) 4.38 (q, J=7.08 Hz, 2H) 7.97 (d, J=1.22 Hz, 1H) 8.17 (d, J=0.98 Hz, 1H); Mass Spectrum MH⁺ 208.

### Step (2): Synthesis of 4-formyl-benzoic acid tert-butyl ester

4-Formylbenzoic acid was suspended in 60 mL of benzene, and the mixture was placed under a nitrogen atmosphere. The mixture was brought to reflux and N,N-dimethylformamide di-tert-butylacetal was added dropwise via an addition funnel over 45 minutes. The yellowish suspension gradually turned golden yellow and became a homogeneous solution. The solution was refluxed an additional 60 minutes before stirring overnight at room temperature. The resulting orange solution was diluted to about 100 mL with ethyl acetate (“EtOAc”), and the resulting solution was washed sequentially with water, saturated sodium bicarbonate (2×100 mL), and brine (100 ml). The solvent was removed by rotary evaporation. The resulting amber oil was injected on to a BIOTAGE® Flash 65i (350 g, silica gel) cartridge and purified with a 30-minute gradient of from 5% to 25% v/v EtOAc in heptane. Product fractions were pooled, and the solvent was rotary evaporated. The resulting golden oil was dried under house high vacuum overnight at room temperature to give 3.92 g (57% yield) of 4-formyl-benzoic acid tert-butyl ester as a yellow solid; 1H NMR (400 MHz, DMSO-D6) δ ppm 1.54 (s, 9H) 8.00 (dm, J=8.30, Hz, 2H) 8.07 (dm, J=8.30, 2H) 10.08 (s, 1H); Mass Spectrum MH⁻ 206.

### Step (3) Synthesis of 4[3-(4-tert-butoxycarbonyl-phenyl)-acryloyl]-6-methyl-pyridine-2-carboxylic acid ethyl ester

4-Acetyl-6-methyl-pyridine-2-carboxylic acid ethyl ester (3.01 g) and 4-formyl-benzoic acid tert-butyl ester (3.0 g), each prepared as described above, and piperidine (0.37 g) were combined in ethanol (25 mL). The reaction mixture was heated at reflux overnight and allowed to cool. The reaction mixture was rotary evaporated to dryness and loaded onto a BIOTAGE® column (350 g silica gel) and purified using a solvent gradient of from 5% to 75% v/v EtOAc in hexane over 40 minutes, from 75% to 100% v/v EtOAc in hexane over 20 minutes, and 30 minutes at 100% EtOAc as the eluent. The appropriate fractions were concentrated to dryness and diethylether was added. A yellow solid was formed upon rotary evaporation of the diethyl ether. The solid was dried under vacuum at 40° C. overnight to give 2.52 g of 4-[3-(4-tert-butoxycarbonyl-phenyl)-acryloyl]-6-methyl-pyridine-2-carboxylic acid ethyl ester; NMR and MS spectra were consistent with the desired product; ¹H NMR (400 MHz, DMSO-D6) δ ppm 1.34 (t, J=7.08 Hz, 3H) 1.53 (d, J=9.52 Hz, 9H) 2.66 (s, 3H) 4.37 (q, J=7.08 Hz, 2H) 7.83 (d, J=15.86 Hz, 1H) 7.97 (m, 5H) 8.18 (d, J=1.22 Hz, 1H) 8.29 (s, 1H); Mass Spectrum MH⁺396.

### Step (4): Synthesis of 4-[3-(4-tert-butoxycarbonyl-phenyl)-propionyl]-6-methyl-pyridine-2-carboxylic acid ethyl ester and 4-[3-(4-tert-Butoxycarbonyl-phenyl)-1-hydroxy-propyl]-6-methyl-pyridine-2-carboxylic acid ethyl ester

4-[3-(4-tert-butoxycarbonyl-phenyl)-acryloyl]-6-methyl-pyridine-2-carboxylic acid ethyl ester (2.52 g), prepared as described above, in tetrahydrofuran (“THF,” 50 mL) was hydrogenated overnight at 50 psi of hydrogen gas using 5% Pd/BaSO₄ (0.25 g) as the catalyst. The mixture was filtered and the filtrate was rotary evaporated to dryness. The residue was purified by column chromatography using a stepwise gradient of from 5% to 100% v/v EtOAc in hexane as the solvent to give 0.41 g of 4-[3-(4-tert-butoxycarbonyl-phenyl)-propionyl]-6-methyl-pyridine-2-carboxylic acid ethyl ester; the NMR and MS spectra were consistent with the desired product; the purity of the product was >98% (by ¹H-NMR); ¹H NMR (400 MHz, DMSO-D6) δ ppm 1.31 (t, J=7.24 Hz, 3H) 1.51 (s, 9H) 2.60 (s, 3H) 2.99 (t, J=7.44 Hz, 2H) 3.47 (t, J=7.44 Hz, 2H) 4.35 (q, J=7.24 Hz, 2H) 7.39 (d, J=7.80 Hz, 2H) 7.80 (d, J=7.80 Hz, 2H) 7.79 (d, J=1.2 Hz, 1H) 8.15 (d, J=1.2 Hz, 1H); Mass Spectrum MH⁺ 398; and 1.05 g of 4-[3-(4-tert-butoxycarbonyl-phenyl)-1-hydroxy-propyl]-6-methyl-pyridine-2-carboxylic acid ethyl ester as an oil; the NMR and MS spectra were consistent with the desired product; the purity of the product was equal to 90% (by ¹H-NMR); ¹H-NMR (400 MHz, DMSO-D6) δ ppm 1.30 (t, J=7.08 Hz, 3H) 1.52 (s, 9H) 1.86 (m, 2H) 2.49 (s, 3H) 2.69 (m, 2H) 4.31 (q, J=7.08 Hz, 2H) 4.59 (m, 1H), 5.60 (d, J=4.6 Hz, 1H) 7.30 (d, J=8.30 Hz, 2H) 7.40 (d, J=1.22 Hz, 1H) 7.78 (d, J=8.54 Hz, 2H) 7.2 (d, J=1.22 Hz, 1H); Mass Spectrum MH⁺ 400.

### Step (5): Synthesis of 4-[3-(4-tert-butoxycarbonyl-phenyl)-1-fluoro-propyl]-6-methyl-pyridine-2-carboxylic acid ethyl ester

A solution of 0.3 g of 4-[3-(4-tert-butoxycarbonyl-phenyl)-1-hydroxy-propyl]-6-methyl-pyridine-2-carboxylic acid ethyl ester, prepared as described above, in dichloromethane (5 mL) was cooled to −78° C. (Acetone/CO₂ bath), (diethylamino)sulfur trifluoride (“DAST”) (0.15 mL) was added, and the reaction mixture stirred cold for 3 hours. A further portion of DAST (0.15 mL) was added, and the reaction mixture was stirred cold for 3 hours. An additional portion of DAST (0.3 mL) was added and the reaction mixture was allowed to warm to −50° C. After 3 hours, the reaction mixture was quenched with water and warmed to room temperature. The organic products were extracted with EtOAc, dried (MgSO₄), and rotary evaporated to dryness. The resulting material (0.33 g) was a 1:1 mixture of 4-[3-(4-tert-butoxycarbonyl-phenyl)-1-fluoro-propyl]-6-methyl-pyridine-2-carboxylic acid ethyl ester and 4-[3(4-tert-butoxycarbonyl-phenyl)-1-hydroxy-propyl]-6-methyl-pyridine-2-carboxylic acid ethyl ester (B) and was used in the next step without further purification; Mass Spectrum MH⁺ 402.

### Step (6): Synthesis of 4-(3-fluoro-3-[2-methyl-6-(3-trifluoromethyl-benzylcarbamoyl)-pyridin-4-yl]-propyl)-benzoic acid tert-butyl ester

A solution of 220 mg of 3-(trifluoromethyl)benzylamine in THF (5 mL) was degassed with a stream of nitrogen for 5 minutes. A 2M solution of trimethylaluminum in toluene (660 μL) was added, and the reaction mixture stirred for 1.5 hours. 4-[3-(4-tert-butoxycarbonyl-phenyl)-1-fluoro-propyl]-6-methyl-pyridine-2-carboxylic acid ethyl ester and 4-[3-(4-tert-butoxycarbonyl-phenyl)-1-hydroxy-propyl]-6-methyl-pyridine-2-carboxylic acid ethyl ester (a mixture), prepared as describe above, (330 mg) was added, and the reaction mixture was stirred at room temperature overnight. To the reaction mixture, stirred vigorously, was added methanol (20 mL). The mixture was concentrated to dryness, and the residue was redissolved in methanol/Toluene and then absorbed onto silica gel. The mixture was purified by BIOTAGE® column chromatography using a gradient of from 5% to 30% v/v EtOAc in hexane over 30 minutes, followed by from 30% to 75% EtOAc in hexane over 30 minutes, followed by from 75% to 100% EtOAc in hexane over 30 minutes. The pure fractions were combined and rotary evaporated to dryness. The residue was dissolved in EtOAc and the solution was filtered through a polytetrafluoroethylene (“PTFE”) filter, and the filtrate was rotary evaporated to dryness to give 0.176 g of 4-{3-fluoro-3-[2-methyl-6-(3-trifluoromethyl-benzylcarbamoyl)-pyridin-4-yl]-propyl}-benzoic acid tert-butyl ester; the NMR and MS spectra were consistent with the desired product; ¹H-NMR (400 MHz, DMSO-D₆) δ ppm 1.51 (s, 9H) 2.16 (m, 2H) 2.56 (s, 3H) 2.76 (t, J=8.05 Hz, 2H) 4.55 (d, J=6.59 Hz, 2H) 5.68 (dm, J=45 Hz 1H) 7.34 (d>J=8.30 Hz, 2H) 7.45 (m, 1H) 7.57 (m, 3H) 7.66 (s, 1H) 7.79 (d, J=8.30 Hz, 2H) 7.82 (s, 1H) 9.34 (t, J=6.34 Hz, 1H); Mass Spectrum MH⁺ 531.

### Step (7): Synthesis of 4-{3-fluoro-3-[2-methyl-6-(3-trifluoromethyl-benzylcarbamoyl)-pyridin-4-yl]-propyl}-benzoic acid

To a solution of 4-{3-fluoro-3-[2-methyl-6-(3-trifluoromethyl-benzylcarbamoyl)-pyridin-4-yl]-propyl}-benzoic acid tert-butyl ester (0.17 g), prepared as describe above, in dichloromethane (3 mL) was added trifluoroacetic acid (“TFA,” 3 mL), and the reaction mixture was stirred at room temperature for 3 hours. The mixture was rotary evaporated to dryness, acetonitrile (4 mL) was added, and the sample was rotary evaporated to dryness. A further portion of acetonitrile (4 mL) was added, and the mixture was rotary evaporated to dryness. Ethyl acetate (1 mL) and hexane (3 mL) were added, and the material was rotary evaporated to dryness to give a solid. The solid was dried under vacuum at 40° C. overnight to give 0.115 g of 4-{3-fluoro-3-[2-methyl-6-(3-trifluoromethyl-benzylcarbamoyl)-pyridin-4-yl]-propyl}-benzoic acid; the NMR and MS spectra were consistent with the desired product; the purity of the product was >90% (by ¹H-NMR). ¹H-NMR (400 MHz, DMSO-D₆) δ ppm 2.17 (d, J=6.83 Hz, 2H) 2.58 (s, 3H) 2.79 (t, J=7.34 Hz, 3H) 4.20 (brs, 1H) 4.58 (d, J=6.34 Hz, 2H) 5.78 (ddd, J=58, 7.93, 4.27 Hz, 1H) 7.37 (d, J=8.30 Hz, 2H) 7.49 (s, 1H) 7.60 (m, 3H) 7.69 (s, 1H) 7.86 (m, 3H) 9.36 (t, J=6.47 Hz, 1H); Mass Spectrum MH⁺ 475.

### EXAMPLE 2

### Preparation of 4-{3,3-difluoro-3-[2-methyl-6-(3-trifluoromethyl-benzylcarbamoyl)-pyridin-4-yl]-propyl}-benzoic acid monotrifluoroacetic acid salt

In a manner similar to that illustrated above in Scheme 2 and described above in Example 1, 4-[3-(4-tert-butoxycarbonyl-phenyl)-propionyl]-6-methyl-pyridine-2-carboxylic acid ethyl ester was converted to 4-[3-(4-tert-butoxycarbonyl-phenyl)-1,1-difluoro-propyl]-6-methyl-pyridine-2-carboxylic acid ethyl ester, which was coupled with 3-(trifluoromethyl)benzylamine to give 4-{3,3-difluoro-3-[2-methyl-6-(3-trifluoromethyl-benzylcarbamoyl)-pyridin-4-yl]-propyl}-benzoic acid tert-butyl ester, which was deprotected to give 4-{3,3-difluoro-3-[2-methyl-6-(3-trifluoromethyl-benzylcarbamoyl)-pyridin-4-yl]-propyl}-benzoic acid monotrifluoroacetic acid salt; The NMR and MS spectra were consistent with the title product. The purity of the product was >98% (by ¹H-NMR). Evidence of 1 mole equivalent of TFA was observed in the fluorine NMR. ¹H-NMR (400 MHz, DMSO-D6) δ ppm 2.60 (m, 2H) 2.62 (s, 3H) 2.79 (m, 2H) 4.59 (d, J=7.5 Hz, 2H) 4.59 (brs, 1H) 7.37 (d, J=8.54 Hz, 2H) 7.60 (m, 3H) 7.69 (m, 2H) 7.83 (ddd, J=8.42, 1.83, 1.71 Hz, 2H) 7.97 (d, J=0.98 Hz, 1H) 9.44 (t, J=6.34 Hz, 1H); MH+ 493.

### EXAMPLE 3

### Preparation of 4-{3-hydroxy-3-[2-methyl-6-(3-trifluoromethyl-benzylcarbamoyl)-pyridin-4-yl]-propyl}-benzoic acid

In a manner similar to that illustrated above in Scheme 2 and described above in Example 1 except that the fluorination step was omitted, 4-[3-(4-tert-butoxycarbonyl-phenyl)-1-hydroxy-propyl]-6-methyl-pyridine-2-carboxylic acid ethyl ester was coupled with 3-trifluoromethylbenzylamine to give 4-{3-hydroxy-3-[2-methyl-6-(3-trifluoromethyl-benzylcarbamoyl)-pyridin-4-yl]-propyl}-benzoic acid.

Solubility of an invention compound was determined according to the procedure described below in Chemical Method 1.

### Chemical Method 1

Solubility of a test compound of the present invention refers to solubility of the compound in a solvent mixture containing ethylene glycol dimethylether (“glyme”) and pH 6.5 aqueous phosphate buffer. The solubility was determined by measuring ultraviolet-visible absorbance of a saturated solution of the compound and comparing the absorbance value to absorbance values obtained for four standard solutions of the compound having known concentrations plotted on a 4-point ultraviolet-visible standard curve.

First, a saturated solution of the test compound of the present invention was prepared by adding from about 2- to about 5-mg of the compound to a 96-well plate and adding a volume of pH 6.5 aqueous phosphate buffer, sonicating the mixture for about 10 minutes, and then allowing it to equilibrate overnight (typically 12 to 24 hours). The saturated solution was visually inspected to ensure the presence of particles, and then filtered to give a filtrate of the saturated solution. The filtrate of the saturated solution and a blank (pH 6.5 aqueous phosphate buffer containing no test compound) were then transferred into separate wells in a 96-well UV-transparent disposable plate, and a small amount of glyme was added to each well to give 5% glyme concentration per well.

Standard solutions of the test compound were prepared by dissolving a known amount of the test compound in a known volume of 75/25 volume/volume (“v/v”) glyme/water, and diluting this solution into four different concentrations in 96-well plates with additional 75/25 v/v glyme/water. Then, aliquots from each of the four standard solutions were also transferred to the 96-well UV-transparent disposable plate containing pH 6.5 aqueous phosphate buffer until the glyme concentration in each well was 5%.

The prepared 96-well plate was transferred to a 96-well ultraviolet-visible plate reader, and the wells were scanned over wavelengths of from 220 nm to 350 nm with a step size of 2 nm. The ultraviolet-visible data (i.e., data generated at the same wavelength) were electronically exported to a spreadsheet and absorbance data for the filtrate of the saturated solution measured at one wavelength (i.e., the data that had the best linear fit as calculated by the highest regression coefficient, typically 0.98 or higher) and the corresponding standards absorbance data measured at the same wavelength were plotted as a standard absorbance-concentration curve based on the Lambert-Beer law. The solubility (i.e., saturation concentration) was then back calculated by comparing the absorbance of the filtrate of the saturated solution to the absorbance of the standard solutions of known concentrations at the same wavelength. The calculated solubility was expressed as in milligrams of test compound in milliliters of solvent. Solubility for compounds of the present invention is shown below in Chemical Method Table 1, in the column labelled “PDS Solubility (mg/L)”.

### Chemical Method Table 1.

| Example No. | PDS Solubility (mg/mL) |
|---|---|
| 1 | 0.027 |
| 2 | 0.004 |
| 3 | N/a^{a} |

^{a}N/a means not available

An invention compound that is a specific inhibitor of MMP-13 may be readily identified by one of ordinary skill in the pharmaceutical or medical arts by assaying a test invention compound for general inhibition of MMP-13 as described below in Biological Method 1. Invention compounds can be further screened with full-length MMP-2, full-length MMP-7, full-length MMP-9, and MMP-14 catalytic domain to determine selectivity of the inhibitors with MMP-13 versus the other MMP enzymes also. Selectivities of the invention compounds for MMP-13 catalytic domain versus another MMP enzyme (full-length or catalytic domain), as determined by dividing the IC₅₀ for the inhibitor with a comparator MMP enzyme by the IC₅₀ of the inhibitor with MMP-13 catalytic domain, are expected to range from 5 to 50,000 fold.

In general, the MMP inhibition assays measure the amount by which a test compound reduces the hydrolysis of a thiopeptolide substrate catalyzed by a matrix metalloproteinase enzyme. Such assays are described in detail by Ye et al., in Biochemistry, 1992;31(45):11231-11235, which is incorporated herein by reference.

Some of the particular methods described below use the catalytic domain of the MMP-13 enzyme, namely matrix metalloproteinase-13 catalytic domain (“MMP-13CD”), rather than the corresponding full-length enzyme, MMP-13. It has been shown previously by Ye Qi-Zhuang, Hupe D., and Johnson L. (Current Medicinal Chemistry, 1996;3:407-418) that inhibitor activity against a catalytic domain of an MMP is predictive of the inhibitor activity against the respective full-length MMP enzyme.

### Biological Method 1

Thiopeptolide substrates show virtually no decomposition or hydrolysis at or below neutral pH in the absence of a matrix metalloproteinase enzyme. A typical thiopeptolide substrate commonly utilized for assays is Ac-Pro-Leu-Gly-thioester-Leu-Leu-Gly-OEt. A 100 μL assay mixture will contain 50 mM of N-2-hydroxyethylpiperazine-N′-2-ethanesulfonic acid buffer (“HEPES,” pH 7.0), 10 mM CaCl₂, 100 μM thiopeptolide substrate, and 1 mM 5,5′-dithio-bis-(2-nitrobenzoic acid) (DTNB). The thiopeptolide substrate concentration may be varied, for example from 10 to 800 μM to obtain Kₘ and K_{cat} values. The change in absorbance at 405 nm is monitored on a Thermo Max microplate reader (molecular Devices, Menlo Park, Calif.) at room temperature (22° C.). The calculation of the amount of hydrolysis of the thiopeptolide substrate is based on E₄₁₂=13600 M⁻¹ cm⁻¹ for the DTNB-derived product 3-carboxy-4-nitrothiophenoxide. Assays are carried out with and without matrix metalloproteinase inhibitor compounds, and the amount of hydrolysis is compared for a determination of inhibitory activity of the test compounds.

Test compounds were evaluated at various concentrations in order to determine their respective IC₅₀ values, the micromolar concentration of compound required to cause a 50% inhibition of catalytic activity of the respective enzyme.

The assay buffer used with MMP-3CD was 50 mM N-morpholinoethane sulfonate (“MES”) at pH 6.0 rather than the HEPES buffer at pH 7.0 described above.

The test described above for the inhibition of MMP-13 may also be adapted and used to determine the ability of the compounds of formula (I) to inhibit the matrix metalloproteases MMP-1, MMP-2, MMP-3, MMP-7, MMP-9, MMP-12, MMP-14, and MMP-17.

The compounds of formula (I), as illustrated by the compounds of Examples 1 to 3, have been shown to be potent inhibitors of MMP-13 catalytic domain. Potencies, as measured by IC₅₀'s expressed in micromolar concentration of the compounds of Examples 1 to 3 with MMP-13 catalytic domain are shown below in Biological Data Table 1 in the columns labelled “MMP-13CD IC₅₀ (μM).”

BIOLOGICAL DATA TABLE 1

| Example | MMP-13CD |
|---|---|
| No. | IC₅₀ (μM) |
| 1 | 0.028, 0.039 |
| 2 | 0.087, 0.11 |
| 3 | 0.062 |

Certain compounds of formula (I) have been assayed with MMP-1 full-length, MMP-2 full-length, MMP-3 catalytic domain, MMP-7 full-length, MMP-8 catalytic domain, MMP-9 full-length, MMP-12 catalytic domain, and MMP-14 catalytic domain. The IC₅₀'s expressed in micromolar concentration of the compounds of Example Nos. (“Ex. No.”) 1 to 3 are as shown below in Biological Data Table 2 in the columns labelled “MMP-1FL IC₅₀ (μM),” “MMP-2FL IC₅₀ (μM),” “MMP-3CD IC₅₀ (μM),” “MMP-7FL IC₅₀ (μM),” “MMP-8CD IC₅₀ (μM),” “MMP-9FL IC₅₀ (μM),” “MMP-12CD IC₅₀ (μM),” and “MMP-14CD IC₅₀ (μM).”

BIOLOGICAL DATA TABLE 2

| Cpd. Ex. | MMP-1FL | MMP-2FL | MMP-3CD | MMP-7FL | MMP-8CD | MMP-9FL | MMP-12CD | MMP-14CD |
|---|---|---|---|---|---|---|---|---|
| No. | IC₅₀ (μM) | IC₅₀ (μM) | IC₅₀ (μM) | IC₅₀ (μM) | IC₅₀ (μM) | IC₅₀ (μM) | IC₅₀ (μM) | IC₅₀ (μM) |
| 1 | >100 | 100 | 11 | 51 | >30 | 51 | >100 | 56 |
| 2 | >100 | >100 | 13 | 1.7 | >30 | 64 | >100 | >100 |
| 3 | >100 | 90 | 6 | <10 | >100 | <10 | >100 | >100 |

As shown be the data in Biological Tables 1 and 2, compounds of the present invention specifically inhibit MMP-13.

Comparison of the IC₅₀'s in Biological Data Tables 1 and 2 show that the invention compounds are potent and specific inhibitors of MMP-13.

Animal models may be used to establish that the instant compounds of formula (I), or a pharmaceutically acceptable salt thereof, would be useful for preventing, treating, and inhibiting cartilage damage, and thus for treating osteoarthritis, for example. Examples of such animal models are described below in Biological Methods 2 and 3.

### Biological Method 2

### Monosodium Iodoacetate-Induced Osteoarthritis in Rat Model of Cartilage Damage (“MIA Rat”)

This model is incorporated by reference from Biological Method 5 of U.S. 2004/0048863 A1.

### Biological Method 3

A transgenic mouse model of MMP-13 mediated osteoarthritis described by Neuhold et al., “Postnatal expression in hyaline cartilage of constitutively active human collagenase-3 (MMP-13) induces osteoarthritis in mice,” J. Clin. Invest., 2001;107(1):35-44, may be used to determine the in vivo effectiveness of an invention compound for treating osteoarthritis.

Still similarly, invention compounds having anti-inflammatory properties may be identified using any one of a number of in vivo animal models of inflammation. For example, for an example of inflammation models, see U.S. Pat. No. 6,329,429, which is incorporated herein by reference.

Still similarly, invention compounds having anti-arthritic properties may be identified using any one of a number of in vivo animal models of arthritis. For example, for an example of arthritis models, see also U.S. Pat. No. 6,329,429.

Still similarly, invention compounds useful for treating heart failure may be identified using any one of a number of in vivo animal models of heart failure. For example, see J. Thomas Peterson et al., Matrix Metalloproteinase Inhibition Attenuates Left Ventricular Modeling and Dysfunction in a Rat Model of Progressive Heart Failure,” Circulation, 2001;103:2303-2309.

Pharmacokinetic properties of the compounds of the present invention, may be determined according to the method of Biological Method 4.

### Biological Method 4

A single 5 mg/kg dose of a test compound is dissolved in 5% N,N-dimethylacetamide/25% propylene glycol/70% 50 mM Tris base and is administered intravenously to a group of 3 Sprague-Dawley rats, and the mean clearance rate of the compound (“IV CL”), expressed in milliliters per minute per kilogram of rat body weight (“mL/min/kg”), and the compound's half-life (“T_{1/2}”), expressed in hours, are determined by conventional means. Further, a single 5 mg/kg oral dose of a test compound is administered in a separate experiment to a group of 3 rats, and the total exposure of blood to the compound is determined by conventional means and reported as the area under the time-concentration of compound curve (“AUC”), expressed in nanograms per hour per milliliter (“ng/hr/mL”).

While the invention has been described and illustrated with reference to certain particular embodiments thereof various adaptations, changes, modifications, substitutions, deletions, or additions of procedures and protocols may be made without departing from the spirit and scope of the invention. It is intended, therefore, that the invention be defined by the scope of the claims that follow and that such claims be interpreted as broadly as is allowed.

All literature and patent references, including patents, patent applications, and patent application publications, cited above are hereby incorporated herein by reference in their entireties and for all purposes.

What is claimed is:

1. A compound of formula (I) 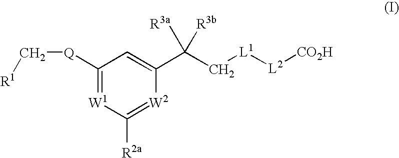
or a pharmaceutically acceptable salt thereof,
wherein:
R¹ is phenyl, or a 5- or 6-membered heteroaryl, wherein the phenyl, or 5- or 6-membered heteroaryl is unsubstituted or substituted on carbon atoms with from 1 to 3 substituent groups T¹;
Q is —(H)N—C(═O)— or —C≡C—;
W¹ and W² independently are N or C—R^{2b};
R^{2a} and R^{2b} independently are H, F, C₁-C₃ alkyl, CF₃, —OH, —O—CH₃, —O—CF₃, —O—CH₂CH₃, or NR^{2c}R^{2d}; or
R^{2a} and R^{2b} are taken together to form a diradical —O—CH₂—O—;
R^{2c} and R^{2d} independently are H, CH₃, or CH₂CH₃;
R^{3a} is F and R^{3b} is H or F; or
R^{3a} is OH and R^{3b} is H; or
R^{3a} and R^{3b} are taken together with the carbon atom to which they are both attached to form C═N—OH;
L¹ is CH₂, O, N(H), S, S(O), S(O)₂, CH₂CH₂, CH₂O, CH₂N(H), CH₂S, CH₂S(O), CH₂S(O)₂, OCH₂, N(H)CH₂, SCH₂, S(O)CH₂, or S(O)₂CH₂, wherein the nitrogen in L¹ is optionally substituted with CH₃;
L² is phenylene, 5- or 6-membered heteroarylene, C₅- or C₆-cycloalkylene, or a 5- or 6-membered heterocycloalkylene, wherein the phenylene or 5- or 6-membered heteroarylene is unsubstituted or substituted on carbon atoms with from 1 to 3 substituents independently selected from the group consisting of CH₃, —OH, —NH₂, F, and CF₃; wherein the C₅- or C₆-cycloalkylene or 5- or 6-membered heterocycloalkylene is unsubstituted or substituted on carbon atoms with from 1 to 3 substituents independently selected from the group consisting of CH₃, —OH, —NH₂, F, CF₃, and oxo; and wherein the 5-membered heteroarylene, 5- or 6-membered heterocycloalkylene is optionally substituted on a nitrogen atom with CH₃; and
each T¹ independently is F, Cl, Br, I, —OH, —OCF₃, CF₃, —CN, —C₁-C₃ alkyl, —O—(C₁-C₃ alkyl), —C(O)—O—(C₁-C₃ alkyl), —C(O)—NH—(C₁-C₃ alkyl), or —S(O)₂—N(H)—(C₁-C₃ alkyl).

2. The compound as in claim 1, or a pharmaceutically acceptable salt thereof, wherein R¹ is phenyl substituted on carbon atoms with 1 or 2 substituents selected from the group consisting of F, —CF₃, —OCH₃, and CH₃ or a 6-membered heteroaryl that is pyridinyl substituted on a carbon atom with OCH₃, Q is —(H)N—C(═O)—, W¹ is N and W² is C—R^{2b}, R^{2a} is H or CH₃, and R^{2b} is H.

3. The compound as in claim 1, wherein W¹ is N and W² is C—R^{2b}, R^{3a} is F and R^{3b} is H, and L¹ is CH₂, CH₂CH₂, or O.

4. The compound as in claim 1, wherein W¹ is N and W² is C—R^{2b}, R^{3a} and R^{3b} are each F, and L¹ is CH₂, CH₂CH₂, or O.

5. The compound as in claim 1, wherein W¹ is N and W² is C—R^{2b}, R^{3a} is F and R^{3b} is H, or R^{3a} and R^{3b} are each F, L¹ is CH₂ or O; and L² is 1,4-phenylene.

6. The compound as in claim 1, wherein W¹ is N and W² is C—R^{2b}, R^{3a} is F and R^{3b} is H, or R^{3a} and R^{3b} are each F, L¹ is CH₂ or O; and L² is 1,4-cyclohexylene.

7. The compound as in claim 1, wherein W¹ is N and W² is C—R^{2b}, R^{3a} is F and R^{3b} is H, or R^{3a} and R^{3b} are each F, L¹ is CH₂ or O; and L² is 6-membered heteroarylene.

8. The compound as in claim 1, selected from the group consisting of:
4-{3-fluoro-3-[2-methyl-6-(3-trifluoromethyl-benzylcarbamoyl)-pyridin-4-yl]-propyl}-benzoic acid;
4-{3,3-difluoro-3-[2-methyl-6-(3-trifluoromethyl-benzylcarbamoyl)-pyridin-4-yl]-propyl}-benzoic acid; and
4-{3-hydroxy-3-[2-methyl-6-(3-trifluoromethyl-benzylcarbamoyl)-pyridin-4-yl]-propyl}-benzoic acid; or
a pharmaceutically acceptable salt thereof.

9. A pharmaceutical composition, comprising the compound as in claim 1, or a pharmaceutically acceptable salt thereof, and a pharmaceutically acceptable excipient.

10. A method of treating osteoarthritis in a mammal, the method comprising administering to a mammal in need thereof a therapeutically effective amount of the compound as in claim 1, or a pharmaceutically acceptable salt thereof.

11. A method of treating rheumatoid arthritis in a mammal, the method comprising administering to a mammal in need thereof a therapeutically effective amount of the compound as in claim 1, or a pharmaceutically acceptable salt thereof.

12. The use of the compound of formula (I), or a pharmaceutically acceptable salt thereof, in the manufacture of a medicament for the treatment of osteoarthritis or rheumatoid arthritis in a mammal.
